(19) United States
(12) Patent Application Publication
(10) Pub. No.: US 2025/0387069 A1
(43) Pub. Date: Dec. 25, 2025

(54) SYSTEMS AND METHODS FOR REMOTE AND LONGITUDINAL MONITORING OF ELECTROENCEPHALOGRAPHIC CHANGES IN GLIOMA PATIENTS

(71) Applicant: UNIVERSITY HEALTH NETWORK, Toronto (CA)

(72) Inventors: Phedias DIAMANDIS, Toronto (CA); Richard SUGDEN, Toronto (CA); Alberto J. LEON, Toronto (CA)

(21) Appl. No.: 19/247,862

(22) Filed: Jun. 24, 2025

Related U.S. Application Data
(60) Provisional application No. 63/663,547, filed on Jun. 24, 2024.

(51) Int. Cl. A61B 5/374; A61B 5/00; A61B 5/378; A61B 5/38; G16H 10/60; G16H 50/20

(52) U.S. Cl. CPC A61B 5/374; A61B 5/0006; A61B 5/378; A61B 5/38; A61B 5/4842; G16H 10/60; G16H 50/20

(57) ABSTRACT

According to an aspect, there is provided systems and methods for remote and longitudinal monitoring of electroencephalographic changes. The method includes remotely collecting electroencephalographic data from an automated session of neurocognitive tasks involving a presentation of audio and/or visual stimuli, time synchronizing the electroencephalographic data to the presentation of the stimuli, processing the electroencephalographic data using an automated pipeline to extract a plurality of features contained in the electroencephalographic data for a patient profile, and performing anomaly detection in the profile of the plurality of features contained in the electroencephalographic data. The feature is associated with a stimuli of the audio and/or visual stimuli and a metric from the electroencephalographic data. The patient profile comprises of a personal baseline.

### CROSS-REFERENCE

The present application claims priority to U.S. provisional patent application No. 63/663,547, titled “SYSTEMS AND METHODS FOR REMOTE AND LONGITUDINAL MONITORING OF ELECTROENCEPHALOGRAPHIC CHANGES IN GLIOMA PATIENTS”, filed on 24 Jun. 2024, the contents of which are incorporated herein by reference.

### FIELD

Embodiments of the present disclosure generally relate to the field of EEG monitoring, and more specifically, embodiments relate to devices, systems and methods for longitudinal EEG monitoring.

### INTRODUCTION

Clinical EEG monitoring can be helpful in the diagnosis, monitoring, and treatment of some conditions or diseases. EEG can be a rich source of biomarkers in numerous neurological conditions; however, clinical EEG protocols may not be well suited as longitudinal disease monitoring tools due to high costs and difficulty to maintain patient engagement. The high cost of the technology makes using clinical EEG unattractive for diagnostic, monitoring, and/or treatment purposes. In particular, device acquisition and staffing costs may restrict its use to clinical settings.

The use of clinical-grade EEG devices may prevent wide-scale adoption of remote and/or longitudinal EEG monitoring, and only focus on diagnosis. The lower electrode densities, and the inability to time-stamp presented stimuli to simultaneous data capture may limit current capabilities of consumer-grade EEG devices. The lack of a platform that leverages such time-stamping stimuli for remote and/or longitudinal EEG measurements for data recording presents a barrier that limits deployment and progress to many exciting biomedical applications and remote care.

Improvement in the field of EEG monitoring is beneficial.

### SUMMARY

Described herein are systems and methods to enable remote and/or longitudinal EEG measurement. Such systems and methods may allow for users to operate EEG tasks independently at home using consumer-grade EEG devices. Furthermore, such systems and methods may unlock the potential to use EEG measurements for ongoing longitudinal monitoring of conditions rather than simply focusing on diagnosis.

The systems and methods described herein may be suitable to carry out a remote neurocognitive task while measuring EEG data from the user from a consumer-grade EEG device. Such approaches may synchronize the EEG data to the presentation of stimuli (e.g., the neurocognitive task). Such approaches may also make available a category of highly informative EEG features (such as event-related potentials (ERPs)) that are extracted based on the presentation of a neurocognitive task.

According to an aspect, there is provided a method for remote and longitudinal monitoring of electroencephalographic changes. The method includes remotely collecting electroencephalographic data from an automated session of neurocognitive tasks involving a presentation of audio and/or visual stimuli, the automated session over a first time period, time synchronizing the electroencephalographic data to the presentation of the stimuli, processing the electroencephalographic data using an automated pipeline to extract a plurality of features contained in the electroencephalographic data for a patient profile, and performing anomaly detection in the profile of the plurality of features contained in the electroencephalographic data. The feature is associated with a stimuli of the audio and/or visual stimuli and a metric from the electroencephalographic data. The patient profile comprises of a personal baseline.

In some embodiments, the plurality of features are the plurality of features listed in Table 1.

In some embodiments, the method further includes time-stamping the electroencephalographic data to synchronize the timing of the presentation of the audio and/or visual stimuli.

In some embodiments, the method further includes using the mean lag time to synchronize the electroencephalographic data and the timing of the presentation of the audio and/or visual stimuli.

In some embodiments, processing the electroencephalographic data includes extracting features comparable to other imaging modalities.

In some embodiments, the method includes repeating the collecting and processing of electroencephalographic data over a plurality of time periods to establish the personal baseline for tracking and detecting changes in the features over the plurality of time periods, and to recapture the features over the plurality of time periods to compare the metrics over the plurality of time periods.

In some embodiments, the method includes remotely collecting additional electroencephalographic data from another automated session of the neurocognitive tasks over a second time period and processing the additional electroencephalographic data using the automated pipeline to extract features from the additional electroencephalographic data, for comparison to the features from the first time period. The feature is associated with the same stimuli of the audio and/or visual stimuli and another metric from the electroencephalographic data.

In some embodiments, the method includes storing, in memory, the profile of the plurality of features along with contextual information.

In some embodiments, the contextual information includes date of collection, a user identifier, and demographic data.

In some embodiments, the method includes detecting habituation-dependent changes and environment-dependent changes in the electroencephalographic data.

In some embodiments, the method includes providing an interactive signal quality check process prior to remotely collecting the electroencephalographic data. The interactive signal quality check provides real time feedback on electroencephalographic signal quality for the session. The interactive signal quality check is tuned to a specific device that generates the electroencephalographic data.

In some embodiments, the method includes providing real time visual feedback on the electroencephalographic signal quality for the session.

In some embodiments, the method includes detecting focal asymmetries in the electroencephalographic data.

In some embodiments, the method includes remotely monitoring a diagnosed pathology over a plurality of time periods using electroencephalographic data.

In some embodiments, the method includes remotely monitoring patient health over a plurality of time periods using electroencephalographic data.

In some embodiments, the method includes tracking the same measurement using one or more features over a plurality of time periods.

In some embodiments, the method includes measuring an improvement using the electroencephalographic data.

In some embodiments, the method includes measuring a treatment response using the electroencephalographic data.

In some embodiments, the method includes detecting a pathology using the electroencephalographic data.

In some embodiments, wherein processing the electroencephalographic data includes identifying event-related potentials in the electroencephalographic data.

According to an aspect, there is provided a system for decentralized electroencephalographic data collection across a plurality of neurocognitive tasks.

According to an aspect, there is provided a system for remote and longitudinal monitoring of electroencephalographic changes. The system includes a user interface application for remotely collecting electroencephalographic data during a plurality of automated sessions that guides neurocognitive tasks while the electroencephalographic data is collected by an electroencephalographic device, the plurality of automated sessions over a plurality of time periods and a server that processes the electroencephalographic data using an automated pipeline to extract a plurality of features contained in the electroencephalographic data over the plurality of time periods, stores the features in a patient profile, generates a personal baseline using the electroencephalographic data, and performs anomaly detection in the profile of the plurality of features contained in the electroencephalographic data. The feature is associated with a stimuli of the audio and/or visual stimuli and a metric from the electroencephalographic data.

In some embodiments, the plurality of features are the plurality of features listed in Table 1.

In some embodiments, the user interface application presents visual stimuli as part of the neurocognitive tasks.

In some embodiments, the user device time-stamps the electroencephalographic data to synchronize it with the visual stimuli.

In some embodiments, the user device uses the mean lag time to synchronize the electroencephalographic data and the timing of the presentation of the audio and/or visual stimuli.

In some embodiments, the electroencephalographic device is a consumer-grade electroencephalographic device.

In some embodiments, the system further includes a display interface to display visual elements corresponding to detected anomalous changes.

According to an aspect, there is provided a non-transitory computer readable medium having recorded thereon statements and instructions for execution by a processing system comprising at least one hardware processor to perform any one of the methods described above.

According to an aspect, there is provided a method for anomaly detection in remotely collected electroencephalographic data. The method includes acquiring electroencephalographic data, processing the electroencephalographic data using an automated pipeline to extract a plurality of features, establishing a personal baseline for tracking and detecting changes in the features over subsequent sessions, the personal baseline including a vector of weights of length equal to the number of features, and each of the weights are a relative relevance assigned to the feature in the personal baseline, repeating the acquisition step and processing step over a plurality of sessions, recapturing the plurality of features over the plurality of sessions to compare the plurality of features over the plurality of sessions, and detecting anomalies in the plurality of features using any combination of one or more of the plurality of features, the detected anomaly indicates presence or change of a medical condition. Each of the features are a measurement of electroencephalographic signals. Each of the features are associated with a position of one or more sensors from which the electroencephalographic data was acquired. The features are associated with a visual and/or auditory stimulus and/or with a continuous task that is executed during acquisition of the electroencephalographic data.

In some embodiments, the plurality of features are associated with an algorithm for processing the EEG signals.

In some embodiments, the plurality of features are the plurality of features listed in Table 1.

In some embodiments, the method includes remotely collecting the electroencephalographic data from an automated session of neurocognitive tasks involving a presentation of the audio and/or visual stimuli and/or the continuous tasks, the automated session over a first time period, time synchronizing the electroencephalographic data to the presentation of the audio and/or visual stimuli and/or the continuous tasks, and processing the electroencephalographic data using the automated pipeline, either locally in an electronic device or remotely in a remote server.

In some embodiments, the medical condition is any type of tumour in the brain regardless of its cell-of-origin.

According to an aspect, there is provided a system for anomaly detection in remotely collected electroencephalographic data. The system includes a measuring device configured to capture electroencephalographic data comprising magnitudes of electric potentials over time in a scalp by using four or more discrete sensors and a user device including a processor, non-volatile data storage, a screen, and audio speakers. The user device including a user interface application for guiding a user during an onboarding process and presenting audio and/or visual stimuli or continuous tasks as part of a neurocognitive task. The user device is configured for wireless communication with the measuring device to receive the magnitudes of the electric potentials over time in the scalp by using the four or more discrete sensors. The user device is configured to synchronize the electroencephalographic data with the presentation of the audio and/or visual stimuli and/or the continuous tasks or transmit the electroencephalographic data to a remote server for synchronization. Synchronization of the electroencephalographic data includes adding time-stamps and labels to the electroencephalographic data. The onboarding process includes checking pairing between the measuring device and the user device, checking battery level of the measuring device, diagnosing quality of acquired signal in real time, and prompting the user to re-fit the four or more discrete sensors as needed.

In some embodiments, the screen includes at least one of a digital screen integrated into the user device or an external screen that the user device is in communication with.

In some embodiments, the user device is one or more of a desktop computer, a laptop computer, a tablet or a smartphone.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 shows a system that can carry out the EEG collection and processing, according to some embodiments.

FIG. 2A shows a process diagram for a method of collecting and processing EEG data, according to some embodiments.

FIG. 2B shows a process diagram for another method of collecting and processing EEG data, according to some embodiments.

FIG. 3A is a schematic showing a subject wearing the EEG wearable device while seated across a portable pre-programmed laptop with the platform, according to some embodiments.

FIG. 3B illustrates a user interface (UI) for an interactive initial signal quality check process, according to some embodiments.

FIG. 3C illustrates a sequence of neurocognitive tasks employed in a standard session (top), a diagram of visual oddball paradigm where oddball and standard stimuli are presented (bottom left), and a diagram sample of a typical P300 waveform evoked by the oddball stimuli (bottom right), according to some embodiments.

FIG. 3D illustrates a standard workflow for remote data collection using the presented system, according to some embodiments.

FIG. 4A shows paired violin plots of alpha activity of periods of eyes-closed and eyes-open in healthy controls, according to some embodiments.

FIG. 4B summarizes the significant changes in power spectra frequency bands changes from eyes-closed to eyes-open resting state conditions by brain regions in control subjects, according to some embodiments.

FIG. 4C shows averaged ERP plots for each electrode, according to some embodiments.

FIG. 4D shows single-epoch minimum-distance to the mean classifier performance of the oddball and standard stimuli, according to some embodiments.

FIG. 4E shows boxplots comparing the peak TP9 and TP10 amplitudes of sessions 1 and 3 (days 1 and 8) combined, and 2 and 4 (days 2 and 9) combined, according to some embodiments.

FIG. 4F shows relative alpha power between in-lab and remote recordings for eyes-closed condition in left temporal-parietal channel from controls, according to some embodiments.

FIG. 4G shows relative theta power between in-lab and remote recordings for eyes-closed condition in left anterior-frontal channel from controls, according to some embodiments.

FIG. 5A shows a visualization of EEG-omic feature space for both patients and controls using UMAP dimensionality reduction, according to some embodiments.

FIG. 5B shows a heatmap showing hierarchical clustering of EEG-omic profiles, according to some embodiments.

FIG. 5C shows a confusion matrix showing proof-of-concept machine learning classification of patients with HGG and controls, according to some embodiments.

FIG. 5D shows a receiver-operator characteristic curve for the classification described by FIG. 5C, according to some embodiments.

FIG. 5E shows a histogram showing highest feature importance values identified by the random forest classifier, according to some embodiments.

FIG. 5F shows bar graphs showing consistent hemispheric-asymmetry of the gamma density across longitudinal sessions, according to some embodiments.

FIG. 5G shows boxplots quantifying the significant asymmetrical decrease in gamma density on the lesional side, according to some embodiments.

FIG. 5H shows boxplots showing the symmetry (non-significance) in controls, according to some embodiments.

FIG. 6 shows timestamping unbiased and blinded radiological changes to the longitudinal monitoring for a patient with a recurrent tumor showing biological tumor evolution over 24 weeks, according to some embodiments.

FIG. 7 shows identified individual spatiotemporal changes in features from longitudinal monitoring a patient corresponding to biological tumor evolution over 17 weeks, according to some embodiments.

FIG. 8 shows alignment of EEG collected from consumer-grade wearables and medical-grade MRI scans, according to some embodiment.

FIG. 9A shows boxplots comparing the latencies of each TP9 and TP10 amplitude peak from sessions 1 and 2 (days 1 and 2) combined, and 3 and 4 (days 8 and 9) combined, according to some embodiments.

FIG. 9B shows boxplots comparing the latencies of each TP9 and TP10 amplitude peak from sessions 1 and 3 (days 1 and 8) combined, and 2 and 4 (days 2 and 9) combined, according to some embodiments.

FIG. 9C shows boxplots comparing the peak TP9 and TP10 amplitudes of sessions 1 and 2 (days 1 and 2) combined, and 3 and 4 (days 8 and 9) combined, according to some embodiments.

FIG. 9D shows boxplots comparing the P300 peak amplitudes measured at the TP9 and TP10 electrodes for each remote session from all healthy participants, according to some embodiments.

FIG. 10A shows boxplots comparing the parietal hemispheres' theta/gamma power ratio in patients 002, 003, and 004 and healthy controls, according to some embodiments.

FIG. 10B shows boxplots comparing the frontal hemispheres' alpha/theta power ratio in patients 002, 003, and 004 and healthy controls, according to some embodiments.

FIG. 10C shows boxplots comparing the frontal hemispheres' theta/gamma power ratio in patients 002, 003, and 004 and healthy controls, according to some embodiments.

FIG. 10D shows boxplots comparing the parietal hemispheres' N200 amplitude in patients 001, 002, 003, and 004 and healthy controls, according to some embodiments.

FIG. 11 is a schematic diagram of computing device 1100, according to some embodiments.

### DETAILED DESCRIPTION

A wealth of potential biomarkers across multiple conditions of the brain can be described in electroencephalography (EEG) data. The high cost of the technology, such as for example due to device acquisition and staffing costs, has restricted its use to diagnostic applications within clinical settings.

Described herein are devices, systems, and methods to provide for remote and/or longitudinal monitoring of electroencephalographic (EEG) changes in users. In particular, the devices, systems, and methods described herein can provide users with guidance through a neurocognitive task while collecting EEG data from the user. The devices, systems, and methods can also synchronize time-stamps in EEG data with the presentation of content to the user. Such devices, systems, and methods may be suitable to enable remote monitoring of EEG data from users using, for example, consumer-grade wearable EEG devices.

Advantages of the systems and methods described herein include that they may be suitable for use with consumer-grade wearable EEG systems to, for example, understand postnatal brain development and monitor neurological diseases remotely. The system may implement a platform for decentralized EEG data collection and can determine, for example, stereotypical and asymmetric EEG patterns in healthy controls and neurologically diseased patients (e.g., post-operative high-grade glioma patients), respectively.

The systems and methods described herein can use consumer-grade EEG and may be usable for monitoring, diagnosis and more. Consumer-grade EEG systems can be advantageous because they are portable, cost effective, and can capture key EEG analysis metrics comparable to clinical-grade systems. Consumer-grade EEG systems can be used for remote and/or longitudinal spectral resting state EEG data that can compare against in-lab medical grade EEG recordings. The systems and methods described herein can allow patients to operate the task independently at home using wearable devices and can open the door for EEG monitoring applications by possibly reducing the cost of each EEG session by, for example, orders of magnitude.

The systems and methods described herein can include an EEG testing platform that can remotely guide participants through specific tasks, record annotated EEG data, and time-stamp EEG data synchronized to the presentation of visual stimuli for the collection of, for example, event-related potentials (ERPs). The EEG testing platform may be provided as an open-source platform.

The systems and methods described herein may be usable to implement a software suite that allows for the collection of EEG data using consumer-grade wearable, the creation of personalized EEG passports for tracking brain health, and the detection of anomalous changes in the EEG passport over time. When EEG is measured (i.e. the data is collected), the time stamping of stimuli can allow temporal alignment of the data to the tasks during which it was collected. This alignment can allow for the generation of features that describe the brain state during EEG measurement. These features can be compared across different time periods (e.g., weeks, months, or years) to look for changes. The collected EEG data can be used to create a personalized EEG passport for tracking of brain health. The user's data can be fed into an automated pipeline that can process the EEG data and generate a profile of, for example, ˜2400 quantitative features (e.g., a measurable property or characteristic of an observable phenomenon) to describe the data.

These features can then be stored in a local database (or elsewhere) alongside information containing, for example, the date of collection, user ID, and other demographic details. The EEG passport can be implemented using an analytical approach for detecting anomalous changes in the EEG passport over time. Anomalies may refer to deviations exhibited by the EEG data (e.g., within the EEG features) as compared to “healthy” or template data or it may refer to deviations exhibited from the user's own baseline data (e.g., identified through longitudinal monitoring). A personal baseline can mean one or more EEG passports generated at a time point that will be used as a point of reference. For example, before treatment can serve as a baseline for post-treatment. Alternatively, any time a healthy subject is deemed healthy can serve as part of their baseline. Then they can track to see if their brain data deviates from that baseline. The baseline can be the average of one or more EEG passports that they take at the baseline time point. By generating these profiles on, for example, consecutive days or weeks, a baseline can be established that can allow for long-term tracking and potential detection of anomalous changes. Importantly, the wide variety of EEG features may make the system capable of handling the specific characteristics of each patient, such as tumour location.

The systems and methods described herein can monitor, for example, brain tumour recurrence by performing anomaly detection in a set of ˜2400 EEG features (e.g., an EEG passport) contained in the EEG data acquired longitudinally (e.g., weekly sessions) and remotely (e.g., from each patient's home). The anomalies in the EEG features that may be detected may coincide with radiological findings of tumour recurrence.

The following table provides a non-limiting examples of some EEG features. These features include the specific electrode location (Electrodes), the frequency range of the EEG response (Frequency Range), the stimulus provided (if any) in the task (Stimulus), and the continuous task (Continuous Task). The table also includes the algorithm used to assess the incoming signals (Algorithm). Each feature is also associated with a unique ID (Feature ID). These are example EEG features and other features may used for other embodiments.

TABLE 1
Example features extracted that may be used to define
an EEG passport, according to some embodiments.

| | | | Frequency | | Continuous |
|---|---|---|---|---|---|
| Feature ID | Algorithm | Electrodes | Range | Stimulus | Task |
| AF7open_mean | mean | AF7 | 0.1 Hz-55 Hz | none | eyes-open |
| AF7open_median | median | AF7 | 0.1 Hz-55 Hz | none | eyes-open |
| AF7open_std | standard | AF7 | 0.1 Hz-55 Hz | none | eyes-open |
| | deviation | | | | |
| AF7open_variance | variance | AF7 | 0.1 Hz-55 Hz | none | eyes-open |
| AF7open_kurtosis | kurtosis | AF7 | 0.1 Hz-55 Hz | none | eyes-open |
| AF7open_skewness | skewness | AF7 | 0.1 Hz-55 Hz | none | eyes-open |
| AF7open_min | minimum | AF7 | 0.1 Hz-55 Hz | none | eyes-open |
| AF7open_max | maximum | AF7 | 0.1 Hz-55 Hz | none | eyes-open |
| AF7open_rms | root mean | AF7 | 0.1 Hz-55 Hz | none | eyes-open |
| | square | | | | |
| AF7open_energy | energy | AF7 | 0.1 Hz-55 Hz | none | eyes-open |
| AF7open_arv | average rectified | AF7 | 0.1 Hz-55 Hz | none | eyes-open |
| | value | | | | |
| AF7open_p2p | peak-to-peak | AF7 | 0.1 Hz-55 Hz | none | eyes-open |
| AF7open_zc | zero crossings | AF7 | 0.1 Hz-55 Hz | none | eyes-open |
| AF7open_msa | mean square | AF7 | 0.1 Hz-55 Hz | none | eyes-open |
| | amplitude | | | | |
| AF7closed_mean | mean | AF7 | 0.1 Hz-55 Hz | none | eyes-closed |
| AF7closed_median | median | AF7 | 0.1 Hz-55 Hz | none | eyes-closed |
| AF7closed_std | standard | AF7 | 0.1 Hz-55 Hz | none | eyes-closed |
| | deviation | | | | |
| AF7closed_variance | variance | AF7 | 0.1 Hz-55 Hz | none | eyes-closed |
| AF7closed_kurtosis | kurtosis | AF7 | 0.1 Hz-55 Hz | none | eyes-closed |
| AF7closed_skewness | skewness | AF7 | 0.1 Hz-55 Hz | none | eyes-closed |
| AF7closed_min | minimum | AF7 | 0.1 Hz-55 Hz | none | eyes-closed |
| AF7closed_max | maximum | AF7 | 0.1 Hz-55 Hz | none | eyes-closed |
| AF7closed_rms | root mean | AF7 | 0.1 Hz-55 Hz | none | eyes-closed |
| | square | | | | |
| AF7closed_energy | energy | AF7 | 0.1 Hz-55 Hz | none | eyes-closed |
| AF7closed_arv | average rectified | AF7 | 0.1 Hz-55 Hz | none | eyes-closed |
| | value | | | | |
| AF7closed_p2p | peak-to-peak | AF7 | 0.1 Hz-55 Hz | none | eyes-closed |
| AF7closed_zc | zero crossings | AF7 | 0.1 Hz-55 Hz | none | eyes-closed |
| AF7closed_msa | mean square | AF7 | 0.1 Hz-55 Hz | none | eyes-closed |
| | amplitude | | | | |
| AF7open_avg_{—} | average moving | AF7 | 0.1 Hz-55 Hz | none | eyes-open |
| moving_slope | slope | | | | |
| AF7closed_avg_{—} | average moving | AF7 | 0.1 Hz-55 Hz | none | eyes-closed |
| moving_slope | slope | | | | |
| AF7open_hjorth_{—} | hjorth activity | AF7 | 0.1 Hz-55 Hz | none | eyes-open |
| activity | | | | | |
| AF7open_hjorth_{—} | hjorth mobility | AF7 | 0.1 Hz-55 Hz | none | eyes-open |
| mobility | | | | | |
| AF7open_hjorth_{—} | hjorth complexity | AF7 | 0.1 Hz-55 Hz | none | eyes-open |
| complexity | | | | | |
| AF7closed_hjorth_{—} | hjorth activity | AF7 | 0.1 Hz-55 Hz | none | eyes-closed |
| activity | | | | | |
| AF7closed_hjorth_{—} | hjorth mobility | AF7 | 0.1 Hz-55 Hz | none | eyes-closed |
| mobility | | | | | |
| AF7closed_hjorth_{—} | hjorth complexity | AF7 | 0.1 Hz-55 Hz | none | eyes-closed |
| complexity | | | | | |
| AF7open_ar_{—} | autoregression | AF7 | 0.1 Hz-55 Hz | none | eyes-open |
| coefficient_{—} | | | | | |
| lag_30 | | | | | |
| AF7open_ar_{—} | autoregression | AF7 | 0.1 Hz-55 Hz | none | eyes-open |
| intercept_30 | | | | | |
| AF7open_ar | autoregression | AF7 | 0.1 Hz-55 Hz | none | eyes-open |
| variance_30 | | | | | |
| AF7closed_ar_{—} | autoregression | AF7 | 0.1 Hz-55 Hz | none | eyes-closed |
| coefficient_{—} | | | | | |
| lag_30 | | | | | |
| AF7closed_ar_{—} | autoregression | AF7 | 0.1 Hz-55 Hz | none | eyes-closed |
| intercept_30 | | | | | |
| AF7closed_ar_{—} | autoregression | AF7 | 0.1 Hz-55 Hz | none | eyes-closed |
| variance_30 | | | | | |
| AF7open_abs_{—} | absolute power | AF7 | 0.5 Hz-4 Hz | none | eyes-open |
| delta | | | | | |
| AF7open_abs_{—} | absolute power | AF7 | 4 Hz-8 Hz | none | eyes-open |
| theta | | | | | |
| AF7open_abs_{—} | absolute power | AF7 | 8 Hz-12 Hz | none | eyes-open |
| alpha | | | | | |
| AF7open_abs_{—} | absolute power | AF7 | 12 Hz-30 Hz | none | eyes-open |
| beta | | | | | |
| AF7open_abs_{—} | absolute power | AF7 | 30 Hz-55 Hz | none | eyes-open |
| gamma | | | | | |
| AF7closed_abs_{—} | absolute power | AF7 | 0.5 Hz-4 Hz | none | eyes-closed |
| delta | | | | | |
| AF7closed_abs_{—} | absolute power | AF7 | 4 Hz-8 Hz | none | eyes-closed |
| theta | | | | | |
| AF7closed_abs_{—} | absolute power | AF7 | 8 Hz-12 Hz | none | eyes-closed |
| alpha | | | | | |
| AF7closed_abs_{—} | absolute power | AF7 | 12 Hz-30 Hz | none | eyes-closed |
| beta | | | | | |
| AF7closed_abs_{—} | absolute power | AF7 | 30 Hz-55 Hz | none | eyes-closed |
| gamma | | | | | |
| AF7open_mean_{—} | mean frequency | AF7 | 0.1 Hz-55 Hz | none | eyes-open |
| frequency | | | | | |
| AF7closedmean_{—} | mean frequency | AF7 | 0.1 Hz-55 Hz | none | eyes-closed |
| frequency | | | | | |
| AF7open_density_{—} | spectral density | AF7 | 0.5 Hz-4 Hz | none | eyes-open |
| delta | | | | | |
| AF7open_density_{—} | spectral density | AF7 | 4 Hz-8 Hz | none | eyes-open |
| theta | | | | | |
| AF7open_density_{—} | spectral density | AF7 | 8 Hz-12 Hz | none | eyes-open |
| alpha | | | | | |
| AF7open_density_{—} | spectral density | AF7 | 12 Hz-30 Hz | none | eyes-open |
| beta | | | | | |
| AF7open_density_{—} | spectral density | AF7 | 30 Hz-55 Hz | none | eyes-open |
| gamma | | | | | |
| AF7closed_density_{—} | spectral density | AF7 | 0.5 Hz-4 Hz | none | eyes-closed |
| delta | | | | | |
| AF7closed_density_{—} | spectral density | AF7 | 4 Hz-8 Hz | none | eyes-closed |
| theta | | | | | |
| AF7closed_density_{—} | spectral density | AF7 | 8 Hz-12 Hz | none | eyes-closed |
| alpha | | | | | |
| AF7closed_density_{—} | spectral density | AF7 | 12 Hz-30 Hz | none | eyes-closed |
| beta | | | | | |
| AF7closed_{—} | spectral density | AF7 | 30 Hz-55 Hz | none | eyes-closed |
| density_gamma | | | | | |
| AF7open_{—} | band ratio | AF7 | 0.5 Hz-4 Hz | none | eyes-open |
| delta/theta | | | 4 Hz-8 Hz | | |
| AF7open_{—} | band ratio | AF7 | 0.5 Hz-4 Hz | none | eyes-open |
| delta/alpha | | | 8 Hz-12 Hz | | |
| AF7open_{—} | band ratio | AF7 | 0.5 Hz-4 Hz | none | eyes-open |
| delta/beta | | | 12 Hz-30 Hz | | |
| AF7open_{—} | band ratio | AF7 | 0.5 Hz-4 Hz | none | eyes-open |
| delta/gamma | | | 30 Hz-55 Hz | | |
| AF7open_{—} | band ratio | AF7 | 0.5 Hz-4 Hz | none | eyes-open |
| theta/delta | | | 4 Hz-8 Hz | | |
| AF7open_{—} | band ratio | AF7 | 4 Hz-8 Hz | none | eyes-open |
| theta/alpha | | | 8 Hz-12 Hz | | |
| AF7open_{—} | band ratio | AF7 | 4 Hz-8 Hz | none | eyes-open |
| theta/beta | | | 12 Hz-30 Hz | | |
| AF7open_{—} | band ratio | AF7 | 4 Hz-8 Hz | none | eyes-open |
| theta/gamma | | | 30 Hz-55 Hz | | |
| AF7open_{—} | band ratio | AF7 | 0.5 Hz-4 Hz | none | eyes-open |
| alpha/delta | | | 8 Hz-12 Hz | | |
| AF7open_{—} | band ratio | AF7 | 4 Hz-8 Hz | none | eyes-open |
| alpha/theta | | | 8 Hz-12 Hz | | |
| AF7open_{—} | band ratio | AF7 | 8 Hz-12 Hz | none | eyes-open |
| alpha/beta | | | 12 Hz-30 Hz | | |
| AF7open_{—} | band ratio | AF7 | 8 Hz-12 Hz | none | eyes-open |
| alpha/gamma | | | 30 Hz-55 Hz | | |
| AF7open_{—} | band ratio | AF7 | 0.5 Hz-4 Hz | none | eyes-open |
| beta/delta | | | 12 Hz-30 Hz | | |
| AF7open_{—} | band ratio | AF7 | 4 Hz-8 Hz | none | eyes-open |
| beta/theta | | | 12 Hz-30 Hz | | |
| AF7open_{—} | band ratio | AF7 | 8 Hz-12 Hz | none | eyes-open |
| beta/alpha | | | 12 Hz-30 Hz | | |
| AF7open_{—} | band ratio | AF7 | 12 Hz-30 Hz | none | eyes-open |
| beta/gamma | | | 30 Hz-55 Hz | | |
| AF7open_{—} | band ratio | AF7 | 0.5 Hz-4 Hz | none | eyes-open |
| gamma/delta | | | 30 Hz-55 Hz | | |
| AF7open_{—} | band ratio | AF7 | 4 Hz-8 Hz | none | eyes-open |
| gamma/theta | | | 30 Hz-55 Hz | | |
| AF7open_{—} | band ratio | AF7 | 8 Hz-12 Hz | none | eyes-open |
| gamma/alpha | | | 30 Hz-55 Hz | | |
| AF7open_{—} | band ratio | AF7 | 12 Hz-30 Hz | none | eyes-open |
| gamma/beta | | | 30 Hz-55 Hz | | |
| AF7closed_{—} | band ratio | AF7 | 0.5 Hz-4 Hz | none | eyes-closed |
| delta/theta | | | 4 Hz-8 Hz | | |
| AF7closed_{—} | band ratio | AF7 | 0.5 Hz-4 Hz | none | eyes-closed |
| delta/alpha | | | 8 Hz-12 Hz | | |
| AF7closed_{—} | band ratio | AF7 | 0.5 Hz-4 Hz | none | eyes-closed |
| delta/beta | | | 12 Hz-30 Hz | | |
| AF7closed_{—} | band ratio | AF7 | 0.5 Hz-4 Hz | none | eyes-closed |
| delta/gamma | | | 30 Hz-55 Hz | | |
| AF7closed_{—} | band ratio | AF7 | 0.5 Hz-4 Hz | none | eyes-closed |
| theta/delta | | | 4 Hz-8 Hz | | |
| AF7closed_{—} | band ratio | AF7 | 4 Hz-8 Hz | none | eyes-closed |
| theta/alpha | | | 8 Hz-12 Hz | | |
| AF7closed_{—} | band ratio | AF7 | 4 Hz-8 Hz | none | eyes-closed |
| theta/beta | | | 12 Hz-30 Hz | | |
| AF7closed_{—} | band ratio | AF7 | 4 Hz-8 Hz | none | eyes-closed |
| theta/gamma | | | 30 Hz-55 Hz | | |
| AF7closed_{—} | band ratio | AF7 | 0.5 Hz-4 Hz | none | eyes-closed |
| alpha/delta | | | 8 Hz-12 Hz | | |
| AF7closed_{—} | band ratio | AF7 | 4 Hz-8 Hz | none | eyes-closed |
| alpha/theta | | | 8 Hz-12 Hz | | |
| AF7closed_{—} | band ratio | AF7 | 8 Hz-12 Hz | none | eyes-closed |
| alpha/beta | | | 12 Hz-30 Hz | | |
| AF7closed_{—} | band ratio | AF7 | 8 Hz-12 Hz | none | eyes-closed |
| alpha/gamma | | | 30 Hz-55 Hz | | |
| AF7closed_{—} | band ratio | AF7 | 0.5 Hz-4 Hz | none | eyes-closed |
| beta/delta | | | 12 Hz-30 Hz | | |
| AF7closed_{—} | band ratio | AF7 | 4 Hz-8 Hz | none | eyes-closed |
| beta/theta | | | 12 Hz-30 Hz | | |
| AF7closed_{—} | band ratio | AF7 | 8 Hz-12 Hz | none | eyes-closed |
| beta/alpha | | | 12 Hz-30 Hz | | |
| AF7closed_{—} | band ratio | AF7 | 12 Hz-30 Hz | none | eyes-closed |
| beta/gamma | | | 30 Hz-55 Hz | | |
| AF7closed_{—} | band ratio | AF7 | 0.5 Hz-4 Hz | none | eyes-closed |
| gamma/delta | | | 30 Hz-55 Hz | | |
| AF7closed_{—} | band ratio | AF7 | 4 Hz-8 Hz | none | eyes-closed |
| gamma/theta | | | 30 Hz-55 Hz | | |
| AF7closed_{—} | band ratio | AF7 | 8 Hz-12 Hz | none | eyes-closed |
| gamma/alpha | | | 30 Hz-55 Hz | | |
| AF7closed_{—} | band ratio | AF7 | 12 Hz-30 Hz | none | eyes-closed |
| gamma/beta | | | 30 Hz-55 Hz | | |
| AF8open_mean | mean | AF8 | 0.1 Hz-55 Hz | none | eyes-open |
| AF8open_median | median | AF8 | 0.1 Hz-55 Hz | none | eyes-open |
| AF8open_std | standard | AF8 | 0.1 Hz-55 Hz | none | eyes-open |
| | deviation | | | | |
| AF8open_variance | variance | AF8 | 0.1 Hz-55 Hz | none | eyes-open |
| AF8open_kurtosis | kurtosis | AF8 | 0.1 Hz-55 Hz | none | eyes-open |
| AF8open_skewness | skewness | AF8 | 0.1 Hz-55 Hz | none | eyes-open |
| AF8open_min | minimum | AF8 | 0.1 Hz-55 Hz | none | eyes-open |
| AF8open_max | maximum | AF8 | 0.1 Hz-55 Hz | none | eyes-open |
| AF8open_rms | root mean | AF8 | 0.1 Hz-55 Hz | none | eyes-open |
| | square | | | | |
| AF8open_energy | energy | AF8 | 0.1 Hz-55 Hz | none | eyes-open |
| AF8open_arv | average rectified | AF8 | 0.1 Hz-55 Hz | none | eyes-open |
| | value | | | | |
| AF8open_p2p | peak-to-peak | AF8 | 0.1 Hz-55 Hz | none | eyes-open |
| AF8open_zc | zero crossings | AF8 | 0.1 Hz-55 Hz | none | eyes-open |
| AF8open_msa | mean square | AF8 | 0.1 Hz-55 Hz | none | eyes-open |
| | amplitude | | | | |
| AF8closed_mean | mean | AF8 | 0.1 Hz-55 Hz | none | eyes-closed |
| AF8closed_median | median | AF8 | 0.1 Hz-55 Hz | none | eyes-closed |
| AF8closed_std | standard | AF8 | 0.1 Hz-55 Hz | none | eyes-closed |
| | deviation | | | | |
| AF8closed_variance | variance | AF8 | 0.1 Hz-55 Hz | none | eyes-closed |
| AF8closed_kurtosis | kurtosis | AF8 | 0.1 Hz-55 Hz | none | eyes-closed |
| AF8closed_skewness | skewness | AF8 | 0.1 Hz-55 Hz | none | eyes-closed |
| AF8closed_min | minimum | AF8 | 0.1 Hz-55 Hz | none | eyes-closed |
| AF8closed_max | maximum | AF8 | 0.1 Hz-55 Hz | none | eyes-closed |
| AF8closed_rms | root mean | AF8 | 0.1 Hz-55 Hz | none | eyes-closed |
| | square | | | | |
| AF8closed_energy | energy | AF8 | 0.1 Hz-55 Hz | none | eyes-closed |
| AF8closed_arv | average rectified | AF8 | 0.1 Hz-55 Hz | none | eyes-closed |
| | value | | | | |
| AF8closed_p2p | peak-to-peak | AF8 | 0.1 Hz-55 Hz | none | eyes-closed |
| AF8closed_zc | zero crossings | AF8 | 0.1 Hz-55 Hz | none | eyes-closed |
| AF8closed_msa | mean square | AF8 | 0.1 Hz-55 Hz | none | eyes-closed |
| | amplitude | | | | |
| AF8open_avg_{—} | average moving | AF8 | 0.1 Hz-55 Hz | none | eyes-open |
| moving_slope | slope | | | | |
| AF8closed_avg_{—} | average moving | AF8 | 0.1 Hz-55 Hz | none | eyes-closed |
| moving_slope | slope | | | | |
| AF8open_hjorth_{—} | hjorth activity | AF8 | 0.1 Hz-55 Hz | none | eyes-open |
| activity | | | | | |
| AF8open_hjorth_{—} | hjorth mobility | AF8 | 0.1 Hz-55 Hz | none | eyes-open |
| mobility | | | | | |
| AF8open_hjorth_{—} | hjorth complexity | AF8 | 0.1 Hz-55 Hz | none | eyes-open |
| complexity | | | | | |
| AF8closed_h | hjorth activity | AF8 | 0.1 Hz-55 Hz | none | eyes-closed |
| jorth_activity | | | | | |
| AF8closed_hjorth_{—} | hjorth mobility | AF8 | 0.1 Hz-55 Hz | none | eyes-closed |
| mobility | | | | | |
| AF8closed_hjorth_{—} | hjorth complexity | AF8 | 0.1 Hz-55 Hz | none | eyes-closed |
| complexity | | | | | |
| AF8open_ar_{—} | autoregression | AF8 | 0.1 Hz-55 Hz | none | eyes-open |
| coefficient_{—} | | | | | |
| lag_30 | | | | | |
| AF8open_ar_{—} | autoregression | AF8 | 0.1 Hz-55 Hz | none | eyes-open |
| intercept_30 | | | | | |
| AF8open_ar_{—} | autoregression | AF8 | 0.1 Hz-55 Hz | none | eyes-open |
| variance_30 | | | | | |
| AF8closed_ar_{—} | autoregression | AF8 | 0.1 Hz-55 Hz | none | eyes-closed |
| coefficient_{—} | | | | | |
| lag_30 | | | | | |
| AF8closed_ar_{—} | autoregression | AF8 | 0.1 Hz-55 Hz | none | eyes-closed |
| intercept_30 | | | | | |
| AF8closed_ar_{—} | autoregression | AF8 | 0.1 Hz-55 Hz | none | eyes-closed |
| variance_30 | | | | | |
| AF8open_abs_{—} | absolute power | AF8 | 0.5 Hz-4 Hz | none | eyes-open |
| delta | | | | | |
| AF8open_abs_{—} | absolute power | AF8 | 4 Hz-8 Hz | none | eyes-open |
| theta | | | | | |
| AF8open_abs_{—} | absolute power | AF8 | 8 Hz-12 Hz | none | eyes-open |
| alpha | | | | | |
| AF8open_abs_{—} | absolute power | AF8 | 12 Hz-30 Hz | none | eyes-open |
| beta | | | | | |
| AF8open_abs_{—} | absolute power | AF8 | 30 Hz-55 Hz | none | eyes-open |
| gamma | | | | | |
| AF8closed_abs_{—} | absolute power | AF8 | 0.5 Hz-4 Hz | none | eyes-closed |
| delta | | | | | |
| AF8closed_abs_{—} | absolute power | AF8 | 4 Hz-8 Hz | none | eyes-closed |
| theta | | | | | |
| AF8closed_abs_{—} | absolute power | AF8 | 8 Hz-12 Hz | none | eyes-closed |
| alpha | | | | | |
| AF8closed_{—} | absolute power | AF8 | 12 Hz-30 Hz | none | eyes-closed |
| abs_beta | | | | | |
| AF8closed_{—} | absolute power | AF8 | 30 Hz-55 Hz | none | eyes-closed |
| abs_gamma | | | | | |
| AF8open_mean_{—} | mean frequency | AF8 | 0.1 Hz-55 Hz | none | eyes-open |
| frequency | | | | | |
| AF8closed_{—} | mean frequency | AF8 | 0.1 Hz-55 Hz | none | eyes-closed |
| mean_frequency | | | | | |
| AF8open_{—} | spectral density | AF8 | 0.5 Hz-4 Hz | none | eyes-open |
| density_delta | | | | | |
| AF8open_{—} | spectral density | AF8 | 4 Hz-8 Hz | none | eyes-open |
| density_theta | | | | | |
| AF8open_{—} | spectral density | AF8 | 8 Hz-12 Hz | none | eyes-open |
| density_alpha | | | | | |
| AF8open_{—} | spectral density | AF8 | 12 Hz-30 Hz | none | eyes-open |
| density_beta | | | | | |
| AF8open_{—} | spectral density | AF8 | 30 Hz-55 Hz | none | eyes-open |
| density_gamma | | | | | |
| AF8closed_{—} | spectral density | AF8 | 0.5 Hz-4 Hz | none | eyes-closed |
| density_delta | | | | | |
| AF8closed_{—} | spectral density | AF8 | 4 Hz-8 Hz | none | eyes-closed |
| density_theta | | | | | |
| AF8closed_{—} | spectral density | AF8 | 8 Hz-12 Hz | none | eyes-closed |
| density_alpha | | | | | |
| AF8closed_{—} | spectral density | AF8 | 12 Hz-30 Hz | none | eyes-closed |
| density_beta | | | | | |
| AF8closed_{—} | spectral density | AF8 | 30 Hz-55 Hz | none | eyes-closed |
| density_gamma | | | | | |
| AF8open_{—} | band ratio | AF8 | 0.5 Hz-4 Hz | none | eyes-open |
| delta/theta | | | 4 Hz-8 Hz | | |
| AF8open_{—} | band ratio | AF8 | 0.5 Hz-4 Hz | none | eyes-open |
| delta/alpha | | | 8 Hz-12 Hz | | |
| AF8open_{—} | band ratio | AF8 | 0.5 Hz-4 Hz | none | eyes-open |
| delta/beta | | | 12 Hz-30 Hz | | |
| AF8open_{—} | band ratio | AF8 | 0.5 Hz-4 Hz | none | eyes-open |
| delta/gamma | | | 30 Hz-55 Hz | | |
| AF8open_{—} | band ratio | AF8 | 0.5 Hz-4 Hz | none | eyes-open |
| theta/delta | | | 4 Hz-8 Hz | | |
| AF8open_{—} | band ratio | AF8 | 4 Hz-8 Hz | none | eyes-open |
| theta/alpha | | | 8 Hz-12 Hz | | |
| AF8open_{—} | band ratio | AF8 | 4 Hz-8 Hz | none | eyes-open |
| theta/beta | | | 12 Hz-30 Hz | | |
| AF8open_{—} | band ratio | AF8 | 4 Hz-8 Hz | none | eyes-open |
| theta/gamma | | | 30 Hz-55 Hz | | |
| AF8open_{—} | band ratio | AF8 | 0.5 Hz-4 Hz | none | eyes-open |
| alpha/delta | | | 8 Hz-12 Hz | | |
| AF8open_{—} | band ratio | AF8 | 4 Hz-8 Hz | none | eyes-open |
| alpha/theta | | | 8 Hz-12 Hz | | |
| AF8open_{—} | band ratio | AF8 | 8 Hz-12 Hz | none | eyes-open |
| alpha/beta | | | 12 Hz-30 Hz | | |
| AF8open_{—} | band ratio | AF8 | 8 Hz-12 Hz | none | eyes-open |
| alpha/gamma | | | 30 Hz-55 Hz | | |
| AF8open_{—} | band ratio | AF8 | 0.5 Hz-4 Hz | none | eyes-open |
| beta/delta | | | 12 Hz-30 Hz | | |
| AF8open_{—} | band ratio | AF8 | 4 Hz-8 Hz | none | eyes-open |
| beta/theta | | | 12 Hz-30 Hz | | |
| AF8open_{—} | band ratio | AF8 | 8 Hz-12 Hz | none | eyes-open |
| beta/alpha | | | 12 Hz-30 Hz | | |
| AF8open_{—} | band ratio | AF8 | 12 Hz-30 Hz | none | eyes-open |
| beta/gamma | | | 30 Hz-55 Hz | | |
| AF8open_{—} | band ratio | AF8 | 0.5 Hz-4 Hz | none | eyes-open |
| gamma/delta | | | 30 Hz-55 Hz | | |
| AF8open_{—} | band ratio | AF8 | 4 Hz-8 Hz | none | eyes-open |
| gamma/theta | | | 30 Hz-55 Hz | | |
| AF8open_{—} | band ratio | AF8 | 8 Hz-12 Hz | none | eyes-open |
| gamma/alpha | | | 30 Hz-55 Hz | | |
| AF8open_{—} | band ratio | AF8 | 12 Hz-30 Hz | none | eyes-open |
| gamma/beta | | | 30 Hz-55 Hz | | |
| AF8closed_{—} | band ratio | AF8 | 0.5 Hz-4 Hz | none | eyes-closed |
| delta/theta | | | 4 Hz-8 Hz | | |
| AF8closed_{—} | band ratio | AF8 | 0.5 Hz-4 Hz | none | eyes-closed |
| delta/alpha | | | 8 Hz-12 Hz | | |
| AF8closed_{—} | band ratio | AF8 | 0.5 Hz-4 Hz | none | eyes-closed |
| delta/beta | | | 12 Hz-30 Hz | | |
| AF8closed_{—} | band ratio | AF8 | 0.5 Hz-4 Hz | none | eyes-closed |
| delta/gamma | | | 30 Hz-55 Hz | | |
| AF8closed_{—} | band ratio | AF8 | 0.5 Hz-4 Hz | none | eyes-closed |
| theta/delta | | | 4 Hz-8 Hz | | |
| AF8closed_{—} | band ratio | AF8 | 4 Hz-8 Hz | none | eyes-closed |
| theta/alpha | | | 8 Hz-12 Hz | | |
| AF8closed_{—} | band ratio | AF8 | 4 Hz-8 Hz | none | eyes-closed |
| theta/beta | | | 12 Hz-30 Hz | | |
| AF8closed_{—} | band ratio | AF8 | 4 Hz-8 Hz | none | eyes-closed |
| theta/gamma | | | 30 Hz-55 Hz | | |
| AF8closed_{—} | band ratio | AF8 | 0.5 Hz-4 Hz | none | eyes-closed |
| alpha/delta | | | 8 Hz-12 Hz | | |
| AF8closed_{—} | band ratio | AF8 | 4 Hz-8 Hz | none | eyes-closed |
| alpha/theta | | | 8 Hz-12 Hz | | |
| AF8closed_{—} | band ratio | AF8 | 8 Hz-12 Hz | none | eyes-closed |
| alpha/beta | | | 12 Hz-30 Hz | | |
| AF8closed_{—} | band ratio | AF8 | 8 Hz-12 Hz | none | eyes-closed |
| alpha/gamma | | | 30 Hz-55 Hz | | |
| AF8closed_{—} | band ratio | AF8 | 0.5 Hz-4 Hz | none | eyes-closed |
| beta/delta | | | 12 Hz-30 Hz | | |
| AF8closed_{—} | band ratio | AF8 | 4 Hz-8 Hz | none | eyes-closed |
| beta/theta | | | 12 Hz-30 Hz | | |
| AF8closed_{—} | band ratio | AF8 | 8 Hz-12 Hz | none | eyes-closed |
| beta/alpha | | | 12 Hz-30 Hz | | |
| AF8closed_{—} | band ratio | AF8 | 12 Hz-30 Hz | none | eyes-closed |
| beta/gamma | | | 30 Hz-55 Hz | | |
| AF8closed_{—} | band ratio | AF8 | 0.5 Hz-4 Hz | none | eyes-closed |
| gamma/delta | | | 30 Hz-55 Hz | | |
| AF8closed_g | band ratio | AF8 | 4 Hz-8 Hz | none | eyes-closed |
| amma/theta | | | 30 Hz-55 Hz | | |
| AF8closed_{—} | band ratio | AF8 | 8 Hz-12 Hz | none | eyes-closed |
| gamma/alpha | | | 30 Hz-55 Hz | | |
| AF8closed_{—} | band ratio | AF8 | 12 Hz-30 Hz | none | eyes-closed |
| gamma/beta | | | 30 Hz-55 Hz | | |
| TP9open_mean | mean | TP9 | 0.1 Hz-55 Hz | none | eyes-open |
| TP9open_median | median | TP9 | 0.1 Hz-55 Hz | none | eyes-open |
| TP9open_std | standard | TP9 | 0.1 Hz-55 Hz | none | eyes-open |
| | deviation | | | | |
| TP9open_variance | variance | TP9 | 0.1 Hz-55 Hz | none | eyes-open |
| TP9open_kurtosis | kurtosis | TP9 | 0.1 Hz-55 Hz | none | eyes-open |
| TP9open_skewness | skewness | TP9 | 0.1 Hz-55 Hz | none | eyes-open |
| TP9open_min | minimum | TP9 | 0.1 Hz-55 Hz | none | eyes-open |
| TP9open_max | maximum | TP9 | 0.1 Hz-55 Hz | none | eyes-open |
| TP9open_rms | root mean | TP9 | 0.1 Hz-55 Hz | none | eyes-open |
| | square | | | | |
| TP9open_energy | energy | TP9 | 0.1 Hz-55 Hz | none | eyes-open |
| TP9open_arv | average rectified | TP9 | 0.1 Hz-55 Hz | none | eyes-open |
| | value | | | | |
| TP9open_p2p | peak-to-peak | TP9 | 0.1 Hz-55 Hz | none | eyes-open |
| TP9open_zc | zero crossings | TP9 | 0.1 Hz-55 Hz | none | eyes-open |
| TP9open_msa | mean square | TP9 | 0.1 Hz-55 Hz | none | eyes-open |
| | amplitude | | | | |
| TP9closed_mean | mean | TP9 | 0.1 Hz-55 Hz | none | eyes-closed |
| TP9closed_median | median | TP9 | 0.1 Hz-55 Hz | none | eyes-closed |
| TP9closed_std | standard | TP9 | 0.1 Hz-55 Hz | none | eyes-closed |
| | deviation | | | | |
| TP9closed_variance | variance | TP9 | 0.1 Hz-55 Hz | none | eyes-closed |
| TP9closed_kurtosis | kurtosis | TP9 | 0.1 Hz-55 Hz | none | eyes-closed |
| TP9closed_skewness | skewness | TP9 | 0.1 Hz-55 Hz | none | eyes-closed |
| TP9closed_min | minimum | TP9 | 0.1 Hz-55 Hz | none | eyes-closed |
| TP9closed_max | maximum | TP9 | 0.1 Hz-55 Hz | none | eyes-closed |
| TP9closed_rms | root mean | TP9 | 0.1 Hz-55 Hz | none | eyes-closed |
| | square | | | | |
| TP9closed_energy | energy | TP9 | 0.1 Hz-55 Hz | none | eyes-closed |
| TP9closed_arv | average rectified | TP9 | 0.1 Hz-55 Hz | none | eyes-closed |
| | value | | | | |
| TP9closed_p2p | peak-to-peak | TP9 | 0.1 Hz-55 Hz | none | eyes-closed |
| TP9closed_zc | zero crossings | TP9 | 0.1 Hz-55 Hz | none | eyes-closed |
| TP9closed_msa | mean square | TP9 | 0.1 Hz-55 Hz | none | eyes-closed |
| | amplitude | | | | |
| TP9open_avg_{—} | average moving | TP9 | 0.1 Hz-55 Hz | none | eyes-open |
| moving_slope | slope | | | | |
| TP9closed_{—} | average moving | TP9 | 0.1 Hz-55 Hz | none | eyes-closed |
| avg_moving_{—} | slope | | | | |
| slope | | | | | |
| TP9open_hjorth_{—} | hjorth activity | TP9 | 0.1 Hz-55 Hz | none | eyes-open |
| activity | | | | | |
| TP9open_hjorth_{—} | hjorth mobility | TP9 | 0.1 Hz-55 Hz | none | eyes-open |
| mobility | | | | | |
| TP9open_hjorth_{—} | hjorth complexity | TP9 | 0.1 Hz-55 Hz | none | eyes-open |
| complexity | | | | | |
| TP9closed_hjorth_{—} | hjorth activity | TP9 | 0.1 Hz-55 Hz | none | eyes-closed |
| activity | | | | | |
| TP9closed_hjorth_{—} | hjorth mobility | TP9 | 0.1 Hz-55 Hz | none | eyes-closed |
| mobility | | | | | |
| TP9closed_hjorth_{—} | hjorth complexity | TP9 | 0.1 Hz-55 Hz | none | eyes-closed |
| complexity | | | | | |
| TP9open_ar_{—} | autoregression | TP9 | 0.1 Hz-55 Hz | none | eyes-open |
| coefficient_{—} | | | | | |
| lag_30 | | | | | |
| TP9open_ar_{—} | autoregression | TP9 | 0.1 Hz-55 Hz | none | eyes-open |
| intercept_30 | | | | | |
| TP9open_ar_{—} | autoregression | TP9 | 0.1 Hz-55 Hz | none | eyes-open |
| variance_30 | | | | | |
| TP9closed_ar_{—} | autoregression | TP9 | 0.1 Hz-55 Hz | none | eyes-closed |
| coefficient_{—} | | | | | |
| lag_30 | | | | | |
| TP9closed_ar_{—} | autoregression | TP9 | 0.1 Hz-55 Hz | none | eyes-closed |
| intercept_30 | | | | | |
| TP9closed_ar_{—} | autoregression | TP9 | 0.1 Hz-55 Hz | none | eyes-closed |
| variance_30 | | | | | |
| TP9open_abs_{—} | absolute power | TP9 | 0.5 Hz-4 Hz | none | eyes-open |
| delta | | | | | |
| TP9open_abs_{—} | absolute power | TP9 | 4 Hz-8 Hz | none | eyes-open |
| theta | | | | | |
| TP9open_abs_{—} | absolute power | TP9 | 8 Hz-12 Hz | none | eyes-open |
| alpha | | | | | |
| TP9open_abs_{—} | absolute power | TP9 | 12 Hz-30 Hz | none | eyes-open |
| beta | | | | | |
| TP9open_abs_{—} | absolute power | TP9 | 30 Hz-55 Hz | none | eyes-open |
| gamma | | | | | |
| TP9closed_abs_{—} | absolute power | TP9 | 0.5 Hz-4 Hz | none | eyes-closed |
| delta | | | | | |
| TP9closed_abs_{—} | absolute power | TP9 | 4 Hz-8 Hz | none | eyes-closed |
| theta | | | | | |
| TP9closed_abs_{—} | absolute power | TP9 | 8 Hz-12 Hz | none | eyes-closed |
| alpha | | | | | |
| TP9closed_abs_{—} | absolute power | TP9 | 12 Hz-30 Hz | none | eyes-closed |
| beta | | | | | |
| TP9closed_abs_{—} | absolute power | TP9 | 30 Hz-55 Hz | none | eyes-closed |
| gamma | | | | | |
| TP9open_mean_{—} | mean frequency | TP9 | 0.1 Hz-55 Hz | none | eyes-open |
| frequency | | | | | |
| TP9closed_{—} | mean frequency | TP9 | 0.1 Hz-55 Hz | none | eyes-closed |
| mean_frequency | | | | | |
| TP9open_{—} | spectral density | TP9 | 0.5 Hz-4 Hz | none | eyes-open |
| density_delta | | | | | |
| TP9open_{—} | spectral density | TP9 | 4 Hz-8 Hz | none | eyes-open |
| density_theta | | | | | |
| TP9open_{—} | spectral density | TP9 | 8 Hz-12 Hz | none | eyes-open |
| density_alpha | | | | | |
| TP9open_{—} | spectral density | TP9 | 12 Hz-30 Hz | none | eyes-open |
| density_beta | | | | | |
| TP9open_{—} | spectral density | TP9 | 30 Hz-55 Hz | none | eyes-open |
| density_gamma | | | | | |
| TP9closed_{—} | spectral density | TP9 | 0.5 Hz-4 Hz | none | eyes-closed |
| density_delta | | | | | |
| TP9closed_{—} | spectral density | TP9 | 4 Hz-8 Hz | none | eyes-closed |
| density_theta | | | | | |
| TP9closed_{—} | spectral density | TP9 | 8 Hz-12 Hz | none | eyes-closed |
| density_alpha | | | | | |
| TP9closed_{—} | spectral density | TP9 | 12 Hz-30 Hz | none | eyes-closed |
| density_beta | | | | | |
| TP9closed_{—} | spectral density | TP9 | 30 Hz-55 Hz | none | eyes-closed |
| density_gamma | | | | | |
| TP9open_{—} | band ratio | TP9 | 0.5 Hz-4 Hz | none | eyes-open |
| delta/theta | | | 4 Hz-8 Hz | | |
| TP9open_{—} | band ratio | TP9 | 0.5 Hz-4 Hz | none | eyes-open |
| delta/alpha | | | 8 Hz-12 Hz | | |
| TP9open_{—} | band ratio | TP9 | 0.5 Hz-4 Hz | none | eyes-open |
| delta/beta | | | 12 Hz-30 Hz | | |
| TP9open_{—} | band ratio | TP9 | 0.5 Hz-4 Hz | none | eyes-open |
| delta/gamma | | | 30 Hz-55 Hz | | |
| TP9open_{—} | band ratio | TP9 | 0.5 Hz-4 Hz | none | eyes-open |
| theta/delta | | | 4 Hz-8 Hz | | |
| TP9open_{—} | band ratio | TP9 | 4 Hz-8 Hz | none | eyes-open |
| theta/alpha | | | 8 Hz-12 Hz | | |
| TP9open_{—} | band ratio | TP9 | 4 Hz-8 Hz | none | eyes-open |
| theta/beta | | | 12 Hz-30 Hz | | |
| TP9open_{—} | band ratio | TP9 | 4 Hz-8 Hz | none | eyes-open |
| theta/gamma | | | 30 Hz-55 Hz | | |
| TP9open_{—} | band ratio | TP9 | 0.5 Hz-4 Hz | none | eyes-open |
| alpha/delta | | | 8 Hz-12 Hz | | |
| TP9open_{—} | band ratio | TP9 | 4 Hz-8 Hz | none | eyes-open |
| alpha/theta | | | 8 Hz-12 Hz | | |
| TP9open_{—} | band ratio | TP9 | 8 Hz-12 Hz | none | eyes-open |
| alpha/beta | | | 12 Hz-30 Hz | | |
| TP9open_{—} | band ratio | TP9 | 8 Hz-12 Hz | none | eyes-open |
| alpha/gamma | | | 30 Hz-55 Hz | | |
| TP9open_{—} | band ratio | TP9 | 0.5 Hz-4 Hz | none | eyes-open |
| beta/delta | | | 12 Hz-30 Hz | | |
| TP9open_{—} | band ratio | TP9 | 4 Hz-8 Hz | none | eyes-open |
| beta/theta | | | 12 Hz-30 Hz | | |
| TP9open_{—} | band ratio | TP9 | 8 Hz-12 Hz | none | eyes-open |
| beta/alpha | | | 12 Hz-30 Hz | | |
| TP9open_{—} | band ratio | TP9 | 12 Hz-30 Hz | none | eyes-open |
| beta/gamma | | | 30 Hz-55 Hz | | |
| TP9open_{—} | band ratio | TP9 | 0.5 Hz-4 Hz | none | eyes-open |
| gamma/delta | | | 30 Hz-55 Hz | | |
| TP9open_{—} | band ratio | TP9 | 4 Hz-8 Hz | none | eyes-open |
| gamma/theta | | | 30 Hz-55 Hz | | |
| TP9open_{—} | band ratio | TP9 | 8 Hz-12 Hz | none | eyes-open |
| gamma/alpha | | | 30 Hz-55 Hz | | |
| TP9open_{—} | band ratio | TP9 | 12 Hz-30 Hz | none | eyes-open |
| gamma/beta | | | 30 Hz-55 Hz | | |
| TP9closed_{—} | band ratio | TP9 | 0.5 Hz-4 Hz | none | eyes-closed |
| delta/theta | | | 4 Hz-8 Hz | | |
| TP9closed_{—} | band ratio | TP9 | 0.5 Hz-4 Hz | none | eyes-closed |
| delta/alpha | | | 8 Hz-12 Hz | | |
| TP9closed_{—} | band ratio | TP9 | 0.5 Hz-4 Hz | none | eyes-closed |
| delta/beta | | | 12 Hz-30 Hz | | |
| TP9closed_{—} | band ratio | TP9 | 0.5 Hz-4 Hz | none | eyes-closed |
| delta/gamma | | | 30 Hz-55 Hz | | |
| TP9closed_{—} | band ratio | TP9 | 0.5 Hz-4 Hz | none | eyes-closed |
| theta/delta | | | 4 Hz-8 Hz | | |
| TP9closed_{—} | band ratio | TP9 | 4 Hz-8 Hz | none | eyes-closed |
| theta/alpha | | | 8 Hz-12 Hz | | |
| TP9closed_{—} | band ratio | TP9 | 4 Hz-8 Hz | none | eyes-closed |
| theta/beta | | | 12 Hz-30 Hz | | |
| TP9closed_{—} | band ratio | TP9 | 4 Hz-8 Hz | none | eyes-closed |
| theta/gamma | | | 30 Hz-55 Hz | | |
| TP9closed_{—} | band ratio | TP9 | 0.5 Hz-4 Hz | none | eyes-closed |
| alpha/delta | | | 8 Hz-12 Hz | | |
| TP9closed_{—} | band ratio | TP9 | 4 Hz-8 Hz | none | eyes-closed |
| alpha/theta | | | 8 Hz-12 Hz | | |
| TP9closed_{—} | band ratio | TP9 | 8 Hz-12 Hz | none | eyes-closed |
| alpha/beta | | | 12 Hz-30 Hz | | |
| TP9closed_{—} | band ratio | TP9 | 8 Hz-12 Hz | none | eyes-closed |
| alpha/gamma | | | 30 Hz-55 Hz | | |
| TP9closed_{—} | band ratio | TP9 | 0.5 Hz-4 Hz | none | eyes-closed |
| beta/delta | | | 12 Hz-30 Hz | | |
| TP9closed_{—} | band ratio | TP9 | 4 Hz-8 Hz | none | eyes-closed |
| beta/theta | | | 12 Hz-30 Hz | | |
| TP9closed_{—} | band ratio | TP9 | 8 Hz-12 Hz | none | eyes-closed |
| beta/alpha | | | 12 Hz-30 Hz | | |
| TP9closed_{—} | band ratio | TP9 | 12 Hz-30 Hz | none | eyes-closed |
| beta/gamma | | | 30 Hz-55 Hz | | |
| TP9closed_{—} | band ratio | TP9 | 0.5 Hz-4 Hz | none | eyes-closed |
| gamma/delta | | | 30 Hz-55 Hz | | |
| TP9closed_{—} | band ratio | TP9 | 4 Hz-8 Hz | none | eyes-closed |
| gamma/theta | | | 30 Hz-55 Hz | | |
| TP9closed_{—} | band ratio | TP9 | 8 Hz-12 Hz | none | eyes-closed |
| gamma/alpha | | | 30 Hz-55 Hz | | |
| TP9closed_{—} | band ratio | TP9 | 12 Hz-30 Hz | none | eyes-closed |
| gamma/beta | | | 30 Hz-55 Hz | | |
| TP10open_mean | mean | TP10 | 0.1 Hz-55 Hz | none | eyes-open |
| TP10open_median | median | TP10 | 0.1 Hz-55 Hz | none | eyes-open |
| TP10open_std | standard | TP10 | 0.1 Hz-55 Hz | none | eyes-open |
| | deviation | | | | |
| TP10open_variance | variance | TP10 | 0.1 Hz-55 Hz | none | eyes-open |
| TP10open_kurtosis | kurtosis | TP10 | 0.1 Hz-55 Hz | none | eyes-open |
| TP10open_skewness | skewness | TP10 | 0.1 Hz-55 Hz | none | eyes-open |
| TP10open_min | minimum | TP10 | 0.1 Hz-55 Hz | none | eyes-open |
| TP10open_max | maximum | TP10 | 0.1 Hz-55 Hz | none | eyes-open |
| TP10open_rms | root mean | TP10 | 0.1 Hz-55 Hz | none | eyes-open |
| | square | | | | |
| TP10open_energy | energy | TP10 | 0.1 Hz-55 Hz | none | eyes-open |
| TP10open_arv | average rectified | TP10 | 0.1 Hz-55 Hz | none | eyes-open |
| | value | | | | |
| TP10open_p2p | peak-to-peak | TP10 | 0.1 Hz-55 Hz | none | eyes-open |
| TP10open_zc | zero crossings | TP10 | 0.1 Hz-55 Hz | none | eyes-open |
| TP10open_msa | mean square | TP10 | 0.1 Hz-55 Hz | none | eyes-open |
| | amplitude | | | | |
| TP10closed_mean | mean | TP10 | 0.1 Hz-55 Hz | none | eyes-closed |
| TP10closed_median | median | TP10 | 0.1 Hz-55 Hz | none | eyes-closed |
| TP10closed_std | standard | TP10 | 0.1 Hz-55 Hz | none | eyes-closed |
| | deviation | | | | |
| TP10closed_{—} | variance | TP10 | 0.1 Hz-55 Hz | none | eyes-closed |
| variance | | | | | |
| TP10closed_{—} | kurtosis | TP10 | 0.1 Hz-55 Hz | none | eyes-closed |
| kurtosis | | | | | |
| TP10closed_{—} | skewness | TP10 | 0.1 Hz-55 Hz | none | eyes-closed |
| skewness | | | | | |
| TP10closed_min | minimum | TP10 | 0.1 Hz-55 Hz | none | eyes-closed |
| TP10closed_max | maximum | TP10 | 0.1 Hz-55 Hz | none | eyes-closed |
| TP10closed_rms | root mean | TP10 | 0.1 Hz-55 Hz | none | eyes-closed |
| | square | | | | |
| TP10closed_{—} | energy | TP10 | 0.1 Hz-55 Hz | none | eyes-closed |
| energy | | | | | |
| TP10closed_arv | average rectified | TP10 | 0.1 Hz-55 Hz | none | eyes-closed |
| | value | | | | |
| TP10closed_p2p | peak-to-peak | TP10 | 0.1 Hz-55 Hz | none | eyes-closed |
| TP10closed_zc | zero crossings | TP10 | 0.1 Hz-55 Hz | none | eyes-closed |
| TP10closed_msa | mean square | TP10 | 0.1 Hz-55 Hz | none | eyes-closed |
| | amplitude | | | | |
| TP10open_avg_{—} | average moving | TP10 | 0.1 Hz-55 Hz | none | eyes-open |
| moving_slope | slope | | | | |
| TP10closed_avg_{—} | average moving | TP10 | 0.1 Hz-55 Hz | none | eyes-closed |
| moving_slope | slope | | | | |
| TP10open_hjorth_{—} | hjorth activity | TP10 | 0.1 Hz-55 Hz | none | eyes-open |
| activity | | | | | |
| TP10open_hjorth_{—} | hjorth mobility | TP10 | 0.1 Hz-55 Hz | none | eyes-open |
| mobility | | | | | |
| TP10open_hjorth_{—} | hjorth complexity | TP10 | 0.1 Hz-55 Hz | none | eyes-open |
| complexity | | | | | |
| TP10closed_{—} | hjorth activity | TP10 | 0.1 Hz-55 Hz | none | eyes-closed |
| hjorth_activity | | | | | |
| TP10closed_{—} | hjorth mobility | TP10 | 0.1 Hz-55 Hz | none | eyes-closed |
| hjorth_mobility | | | | | |
| TP10closed_{—} | hjorth complexity | TP10 | 0.1 Hz-55 Hz | none | eyes-closed |
| hjorth_{—} | | | | | |
| complexity | | | | | |
| TP10open_ar_{—} | autoregression | TP10 | 0.1 Hz-55 Hz | none | eyes-open |
| coefficient_{—} | | | | | |
| lag_30 | | | | | |
| TP10open_ar_{—} | autoregression | TP10 | 0.1 Hz-55 Hz | none | eyes-open |
| intercept_30 | | | | | |
| TP10open_ar_{—} | autoregression | TP10 | 0.1 Hz-55 Hz | none | eyes-open |
| variance_30 | | | | | |
| TP10closed_ar_{—} | autoregression | TP10 | 0.1 Hz-55 Hz | none | eyes-closed |
| coefficient_{—} | | | | | |
| lag_30 | | | | | |
| TP10closed_ar_{—} | autoregression | TP10 | 0.1 Hz-55 Hz | none | eyes-closed |
| intercept_30 | | | | | |
| TP10closed_ar_{—} | autoregression | TP10 | 0.1 Hz-55 Hz | none | eyes-closed |
| variance_30 | | | | | |
| TP10open_abs_{—} | absolute power | TP10 | 0.5 Hz-4 Hz | none | eyes-open |
| delta | | | | | |
| TP10open_abs_{—} | absolute power | TP10 | 4 Hz-8 Hz | none | eyes-open |
| theta | | | | | |
| TP10open_abs_{—} | absolute power | TP10 | 8 Hz-12 Hz | none | eyes-open |
| alpha | | | | | |
| TP10open_abs_{—} | absolute power | TP10 | 12 Hz-30 Hz | none | eyes-open |
| beta | | | | | |
| TP10open_abs_{—} | absolute power | TP10 | 30 Hz-55 Hz | none | eyes-open |
| gamma | | | | | |
| TP10closed_{—} | absolute power | TP10 | 0.5 Hz-4 Hz | none | eyes-closed |
| abs_delta | | | | | |
| TP10closed_{—} | absolute power | TP10 | 4 Hz-8 Hz | none | eyes-closed |
| abs_theta | | | | | |
| TP10closed_{—} | absolute power | TP10 | 8 Hz-12 Hz | none | eyes-closed |
| abs_alpha | | | | | |
| TP10closed | absolute power | TP10 | 12 Hz-30 Hz | none | eyes-closed |
| abs_beta | | | | | |
| TP10closed_{—} | absolute power | TP10 | 30 Hz-55 Hz | none | eyes-closed |
| abs_gamma | | | | | |
| TP10open_{—} | mean frequency | TP10 | 0.1 Hz-55 Hz | none | eyes-open |
| mean_frequency | | | | | |
| TP10closed_{—} | mean frequency | TP10 | 0.1 Hz-55 Hz | none | eyes-closed |
| mean_frequency | | | | | |
| TP10open_{—} | spectral density | TP10 | 0.5 Hz-4 Hz | none | eyes-open |
| density_delta | | | | | |
| TP10open_{—} | spectral density | TP10 | 4 Hz-8 Hz | none | eyes-open |
| density_theta | | | | | |
| TP10open_{—} | spectral density | TP10 | 8 Hz-12 Hz | none | eyes-open |
| density_alpha | | | | | |
| TP10open_{—} | spectral density | TP10 | 12 Hz-30 Hz | none | eyes-open |
| density_beta | | | | | |
| TP10open_{—} | spectral density | TP10 | 30 Hz-55 Hz | none | eyes-open |
| density_gamma | | | | | |
| TP10closed_{—} | spectral density | TP10 | 0.5 Hz-4 Hz | none | eyes-closed |
| density_delta | | | | | |
| TP10closed_{—} | spectral density | TP10 | 4 Hz-8 Hz | none | eyes-closed |
| density_theta | | | | | |
| TP10closed_{—} | spectral density | TP10 | 8 Hz-12 Hz | none | eyes-closed |
| density_alpha | | | | | |
| TP10closed_{—} | spectral density | TP10 | 12 Hz-30 Hz | none | eyes-closed |
| density_beta | | | | | |
| TP10closed_{—} | spectral density | TP10 | 30 Hz-55 Hz | none | eyes-closed |
| density_gamma | | | | | |
| TP10open_{—} | band ratio | TP10 | 0.5 Hz-4 Hz | none | eyes-open |
| delta/theta | | | 4 Hz-8 Hz | | |
| TP10open_{—} | band ratio | TP10 | 0.5 Hz-4 Hz | none | eyes-open |
| delta/alpha | | | 8 Hz-12 Hz | | |
| TP10open_{—} | band ratio | TP10 | 0.5 Hz-4 Hz | none | eyes-open |
| delta/beta | | | 12 Hz-30 Hz | | |
| TP10open_{—} | band ratio | TP10 | 0.5 Hz-4 Hz | none | eyes-open |
| delta/gamma | | | 30 Hz-55 Hz | | |
| TP10open_{—} | band ratio | TP10 | 0.5 Hz-4 Hz | none | eyes-open |
| theta/delta | | | 4 Hz-8 Hz | | |
| TP10open_{—} | band ratio | TP10 | 4 Hz-8 Hz | none | eyes-open |
| theta/alpha | | | 8 Hz-12 Hz | | |
| TP10open_{—} | band ratio | TP10 | 4 Hz-8 Hz | none | eyes-open |
| theta/beta | | | 12 Hz-30 Hz | | |
| TP10open_{—} | band ratio | TP10 | 4 Hz-8 Hz | none | eyes-open |
| theta/gamma | | | 30 Hz-55 Hz | | |
| TP10open_{—} | band ratio | TP10 | 0.5 Hz-4 Hz | none | eyes-open |
| alpha/delta | | | 8 Hz-12 Hz | | |
| TP10open_{—} | band ratio | TP10 | 4 Hz-8 Hz | none | eyes-open |
| alpha/theta | | | 8 Hz-12 Hz | | |
| TP10open_{—} | band ratio | TP10 | 8 Hz-12 Hz | none | eyes-open |
| alpha/beta | | | 12 Hz-30 Hz | | |
| TP10open_{—} | band ratio | TP10 | 8 Hz-12 Hz | none | eyes-open |
| alpha/gamma | | | 30 Hz-55 Hz | | |
| TP10open_{—} | band ratio | TP10 | 0.5 Hz-4 Hz | none | eyes-open |
| beta/delta | | | 12 Hz-30 Hz | | |
| TP10open_{—} | band ratio | TP10 | 4 Hz-8 Hz | none | eyes-open |
| beta/theta | | | 12 Hz-30 Hz | | |
| TP10open_{—} | band ratio | TP10 | 8 Hz-12 Hz | none | eyes-open |
| beta/alpha | | | 12 Hz-30 Hz | | |
| TP10open_{—} | band ratio | TP10 | 12 Hz-30 Hz | none | eyes-open |
| beta/gamma | | | 30 Hz-55 Hz | | |
| TP10open_{—} | band ratio | TP10 | 0.5 Hz-4 Hz | none | eyes-open |
| gamma/delta | | | 30 Hz-55 Hz | | |
| TP10open_{—} | band ratio | TP10 | 4 Hz-8 Hz | none | eyes-open |
| gamma/theta | | | 30 Hz-55 Hz | | |
| TP10open_{—} | band ratio | TP10 | 8 Hz-12 Hz | none | eyes-open |
| gamma/alpha | | | 30 Hz-55 Hz | | |
| TP10open_{—} | band ratio | TP10 | 12 Hz-30 Hz | none | eyes-open |
| gamma/beta | | | 30 Hz-55 Hz | | |
| TP10closed_{—} | band ratio | TP10 | 0.5 Hz-4 Hz | none | eyes-closed |
| delta/theta | | | 4 Hz-8 Hz | | |
| TP10closed_{—} | band ratio | TP10 | 0.5 Hz-4 Hz | none | eyes-closed |
| delta/alpha | | | 8 Hz-12 Hz | | |
| TP10closed_{—} | band ratio | TP10 | 0.5 Hz-4 Hz | none | eyes-closed |
| delta/beta | | | 12 Hz-30 Hz | | |
| TP10closed_{—} | band ratio | TP10 | 0.5 Hz-4 Hz | none | eyes-closed |
| delta/gamma | | | 30 Hz-55 Hz | | |
| TP10closed_{—} | band ratio | TP10 | 0.5 Hz-4 Hz | none | eyes-closed |
| theta/delta | | | 4 Hz-8 Hz | | |
| TP10closed_{—} | band ratio | TP10 | 4 Hz-8 Hz | none | eyes-closed |
| theta/alpha | | | 8 Hz-12 Hz | | |
| TP10closed_{—} | band ratio | TP10 | 4 Hz-8 Hz | none | eyes-closed |
| theta/beta | | | 12 Hz-30 Hz | | |
| TP10closed_{—} | band ratio | TP10 | 4 Hz-8 Hz | none | eyes-closed |
| theta/gamma | | | 30 Hz-55 Hz | | |
| TP10closed_{—} | band ratio | TP10 | 0.5 Hz-4 Hz | none | eyes-closed |
| alpha/delta | | | 8 Hz-12 Hz | | |
| TP10closed_{—} | band ratio | TP10 | 4 Hz-8 Hz | none | eyes-closed |
| alpha/theta | | | 8 Hz-12 Hz | | |
| TP10closed_{—} | band ratio | TP10 | 8 Hz-12 Hz | none | eyes-closed |
| alpha/beta | | | 12 Hz-30 Hz | | |
| TP10closed_{—} | band ratio | TP10 | 8 Hz-12 Hz | none | eyes-closed |
| alpha/gamma | | | 30 Hz-55 Hz | | |
| TP10closed_{—} | band ratio | TP10 | 0.5 Hz-4 Hz | none | eyes-closed |
| beta/delta | | | 12 Hz-30 Hz | | |
| TP10closed_{—} | band ratio | TP10 | 4 Hz-8 Hz | none | eyes-closed |
| beta/theta | | | 12 Hz-30 Hz | | |
| TP10closed_{—} | band ratio | TP10 | 8 Hz-12 Hz | none | eyes-closed |
| beta/alpha | | | 12 Hz-30 Hz | | |
| TP10closed_{—} | band ratio | TP10 | 12 Hz-30 Hz | none | eyes-closed |
| beta/gamma | | | 30 Hz-55 Hz | | |
| TP10closed_{—} | band ratio | TP10 | 0.5 Hz-4 Hz | none | eyes-closed |
| gamma/delta | | | 30 Hz-55 Hz | | |
| TP10closed_{—} | band ratio | TP10 | 4 Hz-8 Hz | none | eyes-closed |
| gamma/theta | | | 30 Hz-55 Hz | | |
| TP10closed_{—} | band ratio | TP10 | 8 Hz-12 Hz | none | eyes-closed |
| gamma/alpha | | | 30 Hz-55 Hz | | |
| TP10closed_{—} | band ratio | TP10 | 12 Hz-30 Hz | none | eyes-closed |
| gamma/beta | | | 30 Hz-55 Hz | | |
| AF7_p300_{—} | amplitude | AF7 | 1 Hz-30 Hz | visual | NA |
| amplitude | | | | oddball | |
| AF7_p300_{—} | latency | AF7 | 1 Hz-30 Hz | visual | NA |
| latency | | | | oddball | |
| AF7_n200_{—} | amplitude | AF7 | 1 Hz-30 Hz | visual | NA |
| amplitude | | | | oddball | |
| AF7_n200_{—} | latency | AF7 | 1 Hz-30 Hz | visual | NA |
| latency | | | | oddball | |
| AF7_p300_{—} | duration | AF7 | 1 Hz-30 Hz | visual | NA |
| duration | | | | oddball | |
| AF7_p300_auc | area under curve | AF7 | 1 Hz-30 Hz | visual | NA |
| | | | | oddball | |
| AF7_n200_{—} | duration | AF7 | 1 Hz-30 Hz | visual | NA |
| duration | | | | oddball | |
| AF7_n200_auc | area under curve | AF7 | 1 Hz-30 Hz | visual | NA |
| | | | | oddball | |
| AF8_p300_{—} | amplitude | AF8 | 1 Hz-30 Hz | visual | NA |
| amplitude | | | | oddball | |
| AF8_p300_{—} | latency | AF8 | 1 Hz-30 Hz | visual | NA |
| latency | | | | oddball | |
| AF8_n200_{—} | amplitude | AF8 | 1 Hz-30 Hz | visual | NA |
| amplitude | | | | oddball | |
| AF8_n200_{—} | latency | AF8 | 1 Hz-30 Hz | visual | NA |
| latency | | | | oddball | |
| AF8_p300_{—} | duration | AF8 | 1 Hz-30 Hz | visual | NA |
| duration | | | | oddball | |
| AF8_p300_auc | area under curve | AF8 | 1 Hz-30 Hz | visual | NA |
| | | | | oddball | |
| AF8_n200_{—} | duration | AF8 | 1 Hz-30 Hz | visual | NA |
| duration | | | | oddball | |
| AF8_n200_auc | area under curve | AF8 | 1 Hz-30 Hz | visual | NA |
| | | | | oddball | |
| TP9_p300_{—} | amplitude | TP9 | 1 Hz-30 Hz | visual | NA |
| amplitude | | | | oddball | |
| TP9_p300_{—} | latency | TP9 | 1 Hz-30 Hz | visual | NA |
| latency | | | | oddball | |
| TP9_n200_{—} | amplitude | TP9 | 1 Hz-30 Hz | visual | NA |
| amplitude | | | | oddball | |
| TP9_n200_{—} | latency | TP9 | 1 Hz-30 Hz | visual | NA |
| latency | | | | oddball | |
| TP9_p300_{—} | duration | TP9 | 1 Hz-30 Hz | visual | NA |
| duration | | | | oddball | |
| TP9_p300_auc | area under curve | TP9 | 1 Hz-30 Hz | visual | NA |
| | | | | oddball | |
| TP9_n200_{—} | duration | TP9 | 1 Hz-30 Hz | visual | NA |
| duration | | | | oddball | |
| TP9_n200_auc | area under curve | TP9 | 1 Hz-30 Hz | visual | NA |
| | | | | oddball | |
| TP10_p300_{—} | amplitude | TP10 | 1 Hz-30 Hz | visual | NA |
| amplitude | | | | oddball | |
| TP10_p300_{—} | latency | TP10 | 1 Hz-30 Hz | visual | NA |
| latency | | | | oddball | |
| TP10_n200_{—} | amplitude | TP10 | 1 Hz-30 Hz | visual | NA |
| amplitude | | | | oddball | |
| TP10_n200_{—} | latency | TP10 | 1 Hz-30 Hz | visual | NA |
| latency | | | | oddball | |
| TP10_p300_{—} | duration | TP10 | 1 Hz-30 Hz | visual | NA |
| duration | | | | oddball | |
| TP10_p300_auc | area under curve | TP10 | 1 Hz-30 Hz | visual | NA |
| | | | | oddball | |
| TP10_n200_{—} | duration | TP10 | 1 Hz-30 Hz | visual | NA |
| duration | | | | oddball | |
| TP10_n200_auc | area under curve | TP10 | 1 Hz-30 Hz | visual | NA |
| | | | | oddball | |
| open_alpha_{—} | connectivity | all | 8 Hz-12 Hz | none | eyes-open |
| connectivity_{—} | coherence | combinations | | | |
| coh | | | | | |
| closed_alpha_{—} | connectivity | all | 8 Hz-12 Hz | none | eyes-closed |
| connectivity_{—} | coherence | combinations | | | |
| coh | | | | | |
| open_beta_{—} | connectivity | all | 12 Hz-30 Hz | none | eyes-open |
| connectivity_{—} | coherence | combinations | | | |
| coh | | | | | |
| closed_beta_{—} | connectivity | all | 12 Hz-30 Hz | none | eyes-closed |
| connectivity_{—} | coherence | combinations | | | |
| coh | | | | | |
| open_theta_{—} | connectivity | all | 4 Hz-8 Hz | none | eyes-open |
| connectivity_{—} | coherence | combinations | | | |
| coh | | | | | |
| closed_theta_{—} | connectivity | all | 4 Hz-8 Hz | none | eyes-closed |
| connectivity_{—} | coherence | combinations | | | |
| coh | | | | | |
| open_delta_{—} | connectivity | all | 0.5 Hz-4 Hz | none | eyes-open |
| connectivity_{—} | coherence | combinations | | | |
| coh | | | | | |
| closed_delta_{—} | connectivity | all | 0.5 Hz-4 Hz | none | eyes-closed |
| connectivity_{—} | coherence | combinations | | | |
| coh | | | | | |
| open_gamma_{—} | connectivity | all | 30 Hz-55 Hz | none | eyes-open |
| connectivity_{—} | coherence | combinations | | | |
| coh | | | | | |
| closed_gamma_{—} | connectivity | all | 30 Hz-55 Hz | none | eyes-closed |
| connectivity_{—} | coherence | combinations | | | |
| coh | | | | | |
| open_alpha_{—} | connectivity | all | 8 Hz-12 Hz | none | eyes-open |
| connectivity_{—} | coherence | combinations | | | |
| cohy | | | | | |
| closed_alpha_{—} | connectivity | all | 8 Hz-12 Hz | none | eyes-closed |
| connectivity_{—} | coherence | combinations | | | |
| cohy | | | | | |
| open_beta_{—} | connectivity | all | 12 Hz-30 Hz | none | eyes-open |
| connectivity_{—} | coherence | combinations | | | |
| cohy | | | | | |
| closed_beta_{—} | connectivity | all | 12 Hz-30 Hz | none | eyes-closed |
| connectivity_{—} | coherence | combinations | | | |
| cohy | | | | | |
| open_theta_{—} | connectivity | all | 4 Hz-8 Hz | none | eyes-open |
| connectivity_{—} | coherence | combinations | | | |
| cohy | | | | | |
| closed_theta_{—} | connectivity | all | 4 Hz-8 Hz | none | eyes-closed |
| connectivity_{—} | coherence | combinations | | | |
| cohy | | | | | |
| open_delta_{—} | connectivity | all | 0.5 Hz-4 Hz | none | eyes-open |
| connectivity_{—} | coherence | combinations | | | |
| cohy | | | | | |
| closed_delta_{—} | connectivity | all | 0.5 Hz-4 Hz | none | eyes-closed |
| connectivity_{—} | coherence | combinations | | | |
| cohy | | | | | |
| open_gamma_{—} | connectivity | all | 30 Hz-55 Hz | none | eyes-open |
| connectivity_{—} | coherence | combinations | | | |
| cohy | | | | | |
| closed_gamma_{—} | connectivity | all | 30 Hz-55 Hz | none | eyes-closed |
| connectivity_{—} | coherence | combinations | | | |
| cohy | | | | | |
| open_alpha_{—} | connectivity | all | 8 Hz-12 Hz | none | eyes-open |
| connectivity_{—} | imaginary | combinations | | | |
| imcoh | coherence | | | | |
| closed_alpha_{—} | connectivity | all | 8 Hz-12 Hz | none | eyes-closed |
| connectivity_{—} | imaginary | combinations | | | |
| imcoh | coherence | | | | |
| open_beta_{—} | connectivity | all | 12 Hz-30 Hz | none | eyes-open |
| connectivity_{—} | imaginary | combinations | | | |
| imcoh | coherence | | | | |
| closed_beta_{—} | connectivity | all | 12 Hz-30 Hz | none | eyes-closed |
| connectivity_{—} | imaginary | combinations | | | |
| imcoh | coherence | | | | |
| open_theta_{—} | connectivity | all | 4 Hz-8 Hz | none | eyes-open |
| connectivity_{—} | imaginary | combinations | | | |
| imcoh | coherence | | | | |
| closed_theta_{—} | connectivity | all | 4 Hz-8 Hz | none | eyes-closed |
| connectivity_{—} | imaginary | combinations | | | |
| imcoh | coherence | | | | |
| open_delta_{—} | connectivity | all | 0.5 Hz-4 Hz | none | eyes-open |
| connectivity_{—} | imaginary | combinations | | | |
| imcoh | coherence | | | | |
| closed_delta_{—} | connectivity | all | 0.5 Hz-4 Hz | none | eyes-closed |
| connectivity_{—} | imaginary | combinations | | | |
| imcoh | coherence | | | | |
| open_gamma_{—} | connectivity | all | 30 Hz-55 Hz | none | eyes-open |
| connectivity_{—} | imaginary | combinations | | | |
| imcoh | coherence | | | | |
| closed_gamma_{—} | connectivity | all | 30 Hz-55 Hz | none | eyes-closed |
| connectivity_{—} | imaginary | combinations | | | |
| imcoh | coherence | | | | |
| open_alpha_{—} | connectivity | all | 8 Hz-12 Hz | none | eyes-open |
| connectivity_{—} | phase locking | combinations | | | |
| plv | value | | | | |
| closed_alpha_{—} | connectivity | all | 8 Hz-12 Hz | none | eyes-closed |
| connectivity_{—} | phase locking | combinations | | | |
| plv | value | | | | |
| open_beta_{—} | connectivity | all | 12 Hz-30 Hz | none | eyes-open |
| connectivity_{—} | phase locking | combinations | | | |
| plv | value | | | | |
| closed_beta_{—} | connectivity | all | 12 Hz-30 Hz | none | eyes-closed |
| connectivity_{—} | phase locking | combinations | | | |
| plv | value | | | | |
| open_theta_{—} | connectivity | all | 4 Hz-8 Hz | none | eyes-open |
| connectivity_{—} | phase locking | combinations | | | |
| plv | value | | | | |
| closed_theta_{—} | connectivity | all | 4 Hz-8 Hz | none | eyes-closed |
| connectivity_{—} | phase locking | combinations | | | |
| plv | value | | | | |
| open_delta_{—} | connectivity | all | 0.5 Hz-4 Hz | none | eyes-open |
| connectivity_{—} | phase locking | combinations | | | |
| plv | value | | | | |
| closed_delta_{—} | connectivity | all | 0.5 Hz-4 Hz | none | eyes-closed |
| connectivity_{—} | phase locking | combinations | | | |
| plv | value | | | | |
| open_gamma_{—} | connectivity | all | 30 Hz-55 Hz | none | eyes-open |
| connectivity_{—} | phase locking | combinations | | | |
| plv | value | | | | |
| closed_gamma_{—} | connectivity | all | 30 Hz-55 Hz | none | eyes-closed |
| connectivity_{—} | phase locking | combinations | | | |
| plv | value | | | | |
| open_alpha_{—} | connectivity | all | 8 Hz-12 Hz | none | eyes-open |
| connectivity_{—} | corrected | combinations | | | |
| ciplv | imaginary phase | | | | |
| | locking value | | | | |
| closed_alpha_{—} | connectivity | all | 8 Hz-12 Hz | none | eyes-closed |
| connectivity_{—} | corrected | combinations | | | |
| ciplv | imaginary phase | | | | |
| | locking value | | | | |
| open_beta_{—} | connectivity | all | 12 Hz-30 Hz | none | eyes-open |
| connectivity_{—} | corrected | combinations | | | |
| ciplv | imaginary phase | | | | |
| | locking value | | | | |
| closed_beta_{—} | connectivity | all | 12 Hz-30 Hz | none | eyes-closed |
| connectivity_{—} | corrected | combinations | | | |
| ciplv | imaginary phase | | | | |
| | locking value | | | | |
| open_theta_{—} | connectivity | all | 4 Hz-8 Hz | none | eyes-open |
| connectivity_{—} | corrected | combinations | | | |
| ciplv | imaginary phase | | | | |
| | locking value | | | | |
| closed_theta_{—} | connectivity | all | 4 Hz-8 Hz | none | eyes-closed |
| connectivity_{—} | corrected | combinations | | | |
| ciplv | imaginary phase | | | | |
| | locking value | | | | |
| open_delta_{—} | connectivity | all | 0.5 Hz-4 Hz | none | eyes-open |
| connectivity_{—} | corrected | combinations | | | |
| ciplv | imaginary phase | | | | |
| | locking value | | | | |
| closed_delta_{—} | connectivity | all | 0.5 Hz-4 Hz | none | eyes-closed |
| connectivity_{—} | corrected | combinations | | | |
| ciplv | imaginary phase | | | | |
| | locking value | | | | |
| open_gamma_{—} | connectivity | all | 30 Hz-55 Hz | none | eyes-open |
| connectivity_{—} | corrected | combinations | | | |
| ciplv | imaginary phase | | | | |
| | locking value | | | | |
| closed_gamma_{—} | connectivity | all | 30 Hz-55 Hz | none | eyes-closed |
| connectivity_{—} | corrected | combinations | | | |
| ciplv | imaginary phase | | | | |
| | locking value | | | | |
| open_alpha_{—} | connectivity | all | 8 Hz-12 Hz | none | eyes-open |
| connectivity_{—} | pairwise phase | combinations | | | |
| ppc | consistency | | | | |
| closed_alpha_{—} | connectivity | all | 8 Hz-12 Hz | none | eyes-closed |
| connectivity_{—} | pairwise phase | combinations | | | |
| ppc | consistency | | | | |
| open_beta_{—} | connectivity | all | 12 Hz-30 Hz | none | eyes-open |
| connectivity_{—} | pairwise phase | combinations | | | |
| ppc | consistency | | | | |
| closed_beta_{—} | connectivity | all | 12 Hz-30 Hz | none | eyes-closed |
| connectivity_{—} | pairwise phase | combinations | | | |
| ppc | consistency | | | | |
| open_theta_{—} | connectivity | all | 4 Hz-8 Hz | none | eyes-open |
| connectivity_{—} | pairwise phase | combinations | | | |
| ppc | consistency | | | | |
| closed_theta_{—} | connectivity | all | 4 Hz-8 Hz | none | eyes-closed |
| connectivity_{—} | pairwise phase | combinations | | | |
| ppc | consistency | | | | |
| open_delta_{—} | connectivity | all | 0.5 Hz-4 Hz | none | eyes-open |
| connectivity_{—} | pairwise phase | combinations | | | |
| ppc | consistency | | | | |
| closed_delta_{—} | connectivity | all | 0.5 Hz-4 Hz | none | eyes-closed |
| connectivity_{—} | pairwise phase | combinations | | | |
| ppc | consistency | | | | |
| open_gamma_{—} | connectivity | all | 30 Hz-55 Hz | none | eyes-open |
| connectivity_{—} | pairwise phase | combinations | | | |
| ppc | consistency | | | | |
| closed_gamma_{—} | connectivity | all | 30 Hz-55 Hz | none | eyes-closed |
| connectivity_{—} | pairwise phase | combinations | | | |
| ppc | consistency | | | | |
| open_alpha_{—} | connectivity | all | 8 Hz-12 Hz | none | eyes-open |
| connectivity_{—} | phase lag index | combinations | | | |
| pli | | | | | |
| closed_alpha_{—} | connectivity | all | 8 Hz-12 Hz | none | eyes-closed |
| connectivity_{—} | phase lag index | combinations | | | |
| pli | | | | | |
| open_beta_{—} | connectivity | all | 12 Hz-30 Hz | none | eyes-open |
| connectivity_{—} | phase lag index | combinations | | | |
| pli | | | | | |
| closed_beta_{—} | connectivity | all | 12 Hz-30 Hz | none | eyes-closed |
| connectivity_{—} | phase lag index | combinations | | | |
| pli | | | | | |
| open_theta_{—} | connectivity | all | 4 Hz-8 Hz | none | eyes-open |
| connectivity_{—} | phase lag index | combinations | | | |
| pli | | | | | |
| closed_theta_{—} | connectivity | all | 4 Hz-8 Hz | none | eyes-closed |
| connectivity_{—} | phase lag index | combinations | | | |
| pli | | | | | |
| open_delta_{—} | connectivity | all | 0.5 Hz-4 Hz | none | eyes-open |
| connectivity_{—} | phase lag index | combinations | | | |
| pli | | | | | |
| closed_delta_{—} | connectivity | all | 0.5 Hz-4 Hz | none | eyes-closed |
| connectivity_{—} | phase lag index | combinations | | | |
| pli | | | | | |
| open_gamma_{—} | connectivity | all | 30 Hz-55 Hz | none | eyes-open |
| connectivity_{—} | phase lag index | combinations | | | |
| pli | | | | | |
| closed_gamma_{—} | connectivity | all | 30 Hz-55 Hz | none | eyes-closed |
| connectivity_{—} | phase lag index | combinations | | | |
| pli | | | | | |
| open_alpha_{—} | connectivity | all | 8 Hz-12 Hz | none | eyes-open |
| connectivity_{—} | debiased phase | combinations | | | |
| dpli | lag index | | | | |
| closed_alpha_{—} | connectivity | all | 8 Hz-12 Hz | none | eyes-closed |
| connectivity_{—} | debiased phase | combinations | | | |
| dpli | lag index | | | | |
| open_beta_{—} | connectivity | all | 12 Hz-30 Hz | none | eyes-open |
| connectivity_{—} | debiased phase | combinations | | | |
| dpli | lag index | | | | |
| closed_beta_{—} | connectivity | all | 12 Hz-30 Hz | none | eyes-closed |
| connectivity_{—} | debiased phase | combinations | | | |
| dpli | lag index | | | | |
| open_theta_{—} | connectivity | all | 4 Hz-8 Hz | none | eyes-open |
| connectivity_{—} | debiased phase | combinations | | | |
| dpli | lag index | | | | |
| closed_theta_{—} | connectivity | all | 4 Hz-8 Hz | none | eyes-closed |
| connectivity_{—} | debiased phase | combinations | | | |
| dpli | lag index | | | | |
| open_delta_{—} | connectivity | all | 0.5 Hz-4 Hz | none | eyes-open |
| connectivity_{—} | debiased phase | combinations | | | |
| dpli | lag index | | | | |
| closed_delta_{—} | connectivity | all | 0.5 Hz-4 Hz | none | eyes-closed |
| connectivity_{—} | debiased phase | combinations | | | |
| dpli | lag index | | | | |
| open_gamma_{—} | connectivity | all | 30 Hz-55 Hz | none | eyes-open |
| connectivity_{—} | debiased phase | combinations | | | |
| dpli | lag index | | | | |
| closed_gamma_{—} | connectivity | all | 30 Hz-55 Hz | none | eyes-closed |
| connectivity_{—} | debiased phase | combinations | | | |
| dpli | lag index | | | | |
| open_alpha_{—} | connectivity | all | 8 Hz-12 Hz | none | eyes-open |
| connectivity_{—} | weighted phase | combinations | | | |
| wpli | lag index | | | | |
| closed_alpha_{—} | connectivity | all | 8 Hz-12 Hz | none | eyes-closed |
| connectivity_{—} | weighted phase | combinations | | | |
| wpli | lag index | | | | |
| open_beta_{—} | connectivity | all | 12 Hz-30 Hz | none | eyes-open |
| connectivity_{—} | weighted phase | combinations | | | |
| wpli | lag index | | | | |
| closed_beta_{—} | connectivity | all | 12 Hz-30 Hz | none | eyes-closed |
| connectivity_{—} | weighted phase | combinations | | | |
| wpli | lag index | | | | |
| open_theta_{—} | connectivity | all | 4 Hz-8 Hz | none | eyes-open |
| connectivity_{—} | weighted phase | combinations | | | |
| wpli | lag index | | | | |
| closed_theta_{—} | connectivity | all | 4 Hz-8 Hz | none | eyes-closed |
| connectivity_{—} | weighted phase | combinations | | | |
| wpli | lag index | | | | |
| open_delta_{—} | connectivity | all | 0.5 Hz-4 Hz | none | eyes-open |
| connectivity_{—} | weighted phase | combinations | | | |
| wpli | lag index | | | | |
| closed_delta_{—} | connectivity | all | 0.5 Hz-4 Hz | none | eyes-closed |
| connectivity_{—} | weighted phase | combinations | | | |
| wpli | lag index | | | | |
| open_gamma_{—} | connectivity | all | 30 Hz-55 Hz | none | eyes-open |
| connectivity_{—} | weighted phase | combinations | | | |
| wpli | lag index | | | | |
| closed_gamma_{—} | connectivity | all | 30 Hz-55 Hz | none | eyes-closed |
| connectivity_{—} | weighted phase | combinations | | | |
| wpli | lag index | | | | |
| open_alpha_{—} | connectivity | all | 8 Hz-12 Hz | none | eyes-open |
| connectivity_{—} | debiased | combinations | | | |
| wpli2_debiased | weighted phase | | | | |
| | lag index | | | | |
| closed_alpha_{—} | connectivity | all | 8 Hz-12 Hz | none | eyes-closed |
| connectivity_{—} | debiased | combinations | | | |
| wpli2_debiased | weighted phase | | | | |
| | lag index | | | | |
| open_beta_{—} | connectivity | all | 12 Hz-30 Hz | none | eyes-open |
| connectivity_{—} | debiased | combinations | | | |
| wpli2_debiased | weighted phase | | | | |
| | lag index | | | | |
| closed_beta_{—} | connectivity | all | 12 Hz-30 Hz | none | eyes-closed |
| connectivity_{—} | debiased | combinations | | | |
| wpli2_debiased | weighted phase | | | | |
| | lag index | | | | |
| open_theta_{—} | connectivity | all | 4 Hz-8 Hz | none | eyes-open |
| connectivity_{—} | debiased | combinations | | | |
| wpli2_debiased | weighted phase | | | | |
| | lag index | | | | |
| closed_theta_{—} | connectivity | all | 4 Hz-8 Hz | none | eyes-closed |
| connectivity_{—} | debiased | combinations | | | |
| wpli2_debiased | weighted phase | | | | |
| | lag index | | | | |
| open_delta_{—} | connectivity | all | 0.5 Hz-4 Hz | none | eyes-open |
| connectivity_{—} | debiased | combinations | | | |
| wpli2_debiased | weighted phase | | | | |
| | lag index | | | | |
| closed_delta_{—} | connectivity | all | 0.5 Hz-4 Hz | none | eyes-closed |
| connectivity_{—} | debiased | combinations | | | |
| wpli2_debiased | weighted phase | | | | |
| | lag index | | | | |
| open_gamma_{—} | connectivity | all | 30 Hz-55 Hz | none | eyes-open |
| connectivity_{—} | debiased | combinations | | | |
| wpli2_debiased | weighted phase | | | | |
| | lag index | | | | |
| closed_gamma_{—} | connectivity | all | 30 Hz-55 Hz | none | eyes-closed |
| connectivity_{—} | debiased | combinations | | | |
| wpli2_debiased | weighted phase | | | | |
| | lag index | | | | |

Potential applications include remote monitoring of already-diagnosed pathologies (e.g. brain tumour recurrence) which may prevent unnecessary trips to the hospital for more expensive imaging, measurement of treatment response (e.g., to provide early indication of whether a patient is responding well to medication), and/or widespread screening for rare disease (e.g., the ubiquitous collection of EEG data by an institution that widely deploys EEG could lead to early detection of rare pathologies like brain tumours).

The collection of EEG data using consumer-grade wearables can enable at-home testing using low-cost devices. The data collection software can be downloaded onto a computer with only modest computational requirements (e.g., a windows computer, a mobile device, etc.). The user may be guided by a graphical interface showing instructions for how to connect the wearable device to the low-cost computer. Once connected, the user can be given an interactive signal quality check to allow the user to equip and adjust the device accordingly. Once their device is equipped with good signal quality, the user can begin an automated session where simple instructions can be provided to guide the user's attention while the data can be automatically collected.

The platform, according to some embodiments, leverages wearables for decentralized collection of EEG data across various neurocognitive tasks. In some embodiments, the platform can be used to conduct, for example, remote longitudinal weekly recordings in, for example, high-grade glioma patients, demonstrating evolving EEG profiles that may mirror in-hospital MRI surveillance scans. Consumer-grade EEG systems may be able to capture habituation and environment-dependent changes in EEG patterns, detect focal EEG asymmetries in post-operative brain tumor patients, and align longitudinal EEG patterns with changes in MRI surveillance scans. Some embodiments can provide non-invasive and real-time monitoring of brain development and disease evolution.

In neurologically diseased patients (high-grade glioma), for example, some embodiment platforms can enable remote longitudinal weekly recordings in patients spanning 6-8 months which can highlight evolving longitudinal EEG profiles that may mirror quarterly in-hospital MRI surveillance scans. Such platforms may be feasible and have healthcare potential of remote and longitudinal monitoring of EEG brain activity across neurological diseased states.

Consumer-grade wearable electroencephalographic (EEG) systems may open potential new avenues for research and investigation into the understanding of the human brain through, for example, postnatal development and neurological disease. Unlike other EEG devices, that may be largely confined to hospitals and research settings, brain wearables are portable, battery-operated, and require just a few minutes for setup without the need for a modality-specific technician. Because of these practical advantages, coupled with significant cost savings, (for example, ˜$200-900 USD/device), these devices may democratize detection and monitoring of neurological pathologies outside of traditional centralized research and healthcare settings. Serial data may also be massively collected to characterize longitudinal evolutions of neuropathology by scaling the collection of EEG data to the remote setting using some embodiment platforms. These patterns, when used in combination with subject-specific baseline recordings, may serve as biomarkers for detection of anomalous changes. Some embodiments use consumer-grade wearables to record meaningful and relevant EEG data in a remote setting, across both health and disease.

Some key EEG analysis metrics that may be used by some embodiments of the platform include event-related potentials (ERP) and resting state power spectral data. Such data can be comparable to clinical-grade EEG systems. Some embodiments may be able to predict stroke severity by comparing the power spectral properties of ischemic stroke patients and healthy controls from three-minute recordings from the same devices. The ability to independently monitor disease at-home, and at a low cost, may enable novel avenues for the real-time and longitudinal study of disease detection and evolution. The platform, according to some embodiments, may be capable of leveraging consumer-grade EEG wearables to longitudinally deploy a battery of neurocognitive tasks (including ERPs) in both research and remote settings. Consistent session-to-session asymmetric patterns may show longitudinal evolution spatially and temporally aligned with disease anatomy and radiological signs of progression.

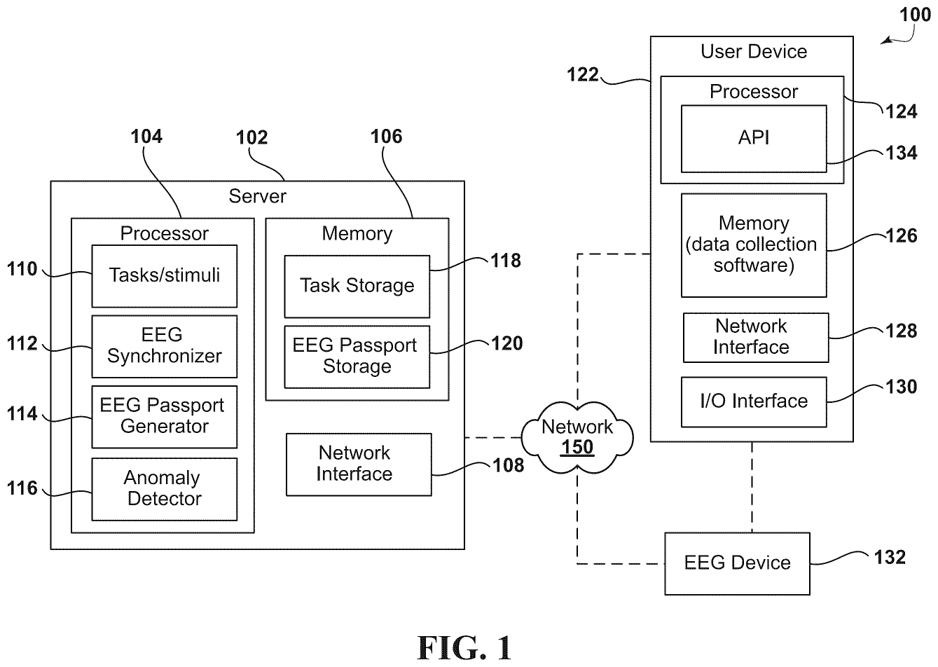

FIG. 1 shows a system 100 that can carry out the EEG collection and processing, according to some embodiments.

The system 100 can include a server 102, a user device 122, and an EEG device 132. The system 100 may be configured to carry out the functions of the platforms described herein. In some embodiments, the server 102 can act as a remote server to which the user device 122 transmits EEG data. In other embodiments, the user device 122 may be configured to carry out some or all of the functionality of the server 102 (e.g., it may conduct some processing of the EEG data before transmission to the server 102). The server 102 may be in communication with the user device 122 and the EEG device 132 over a network 150. In other embodiments, the EEG device 132 may connect to the server 102 through the user device 122.

The server 102 can include a processor 104, a memory 106, and a network interface 108. In some embodiments, the computing device may further include an I/O interface (not shown). The server 102 may be configured to orchestrate the collection of EEG data using EEG device 132 during the presentation of a neurocognitive task using the user device 122, the creation of personalized EEG passports for tracking brain health, and the detection of anomalous changes in the EEG passport over time.

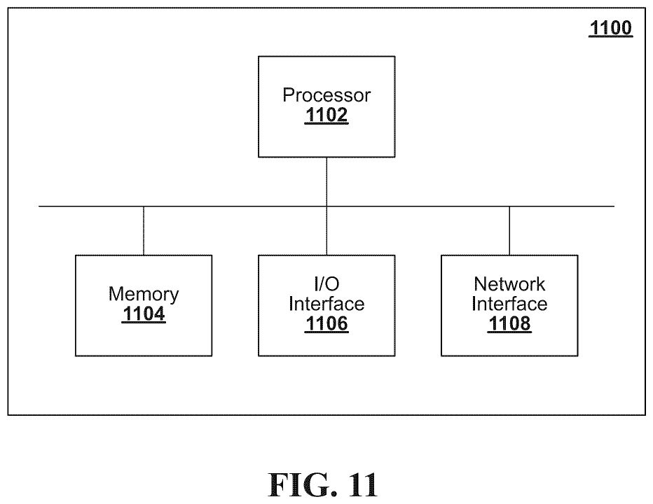

The processor 104 may generally follow the same description as for processor 1102 of FIG. 11 described below. The processor 104 may include a task presenter (e.g. for generating tasks and stimuli) 110, an EEG synchronizer 112, an EEG passport generator 114, and an anomaly detector 116. The processes carried out by the processor 104 may instead be carried out by the processor 124 of the user device 122.

The task presenter 110 may present or cause the presentation of a task via the user device 122. The task presenter 110 may retrieve the relevant neurocognitive task from the memory 106 including the task content (e.g., visual and/or audio content) and metadata (e.g., task protocols which may be used to analyze data). The tasks can include, for example, guidance through activities (e.g., eyes open, eyes closed, etc.), a neurocognitive task (e.g., an oddball task), or other types of content. The system 100 may present task content to the user and use the task metadata to analyze EEG bio-signals generated by the user during the task.

The EEG synchronizer 112 may synchronize EEG data generated by the user to presented content. For example, the EEG data may be synchronized with the presentation of visual stimuli in the task. In some embodiments, the EEG data may be time-stamped to synchronize the data with the presentation of content. The synchronization may label or otherwise associate points and/or regions in the data with relevant presentation information (e.g., regions of the EEG data obtained when the user's eyes were open, regions of the EEG data obtained when the user's eyes were closed, a time when an oddball stimuli was presented to the user). These labels or associations may further be used by the system 100 to process the EEG data. For example, the time-stamp associated with the presentation of an oddball stimuli in the EEG data may be used to extract the reaction time. Other features may be extracted from the EEG data using the labels or associations with the presentation information. Such feature extraction may be carried out using models trained with machine learning. Such feature extraction may be beneficial as it may be used to accurately and precisely assess a user's reaction to neurocognitive tasks. Synchronization may involve, for example, time-stamping the data or it may involve using event-markers that may be derived from expected delays between, for example, the EEG device 132 and the user device 122.

Synchronization may be important when using consumer-grade EEG devices. Particularly when the system 100 may be configured to work with different varieties of consumer-grade EEG devices 132. The data transmission abilities of various EEG devices 132 may differ such that the same neurocognitive tasks may elicit different observed response times from the user when, in fact, the user would have the same response time. Accordingly, synchronizing the EEG data with the presentation of neurocognitive tasks may be important to ensure that results taken on a consumer-grade EEG device can be adequately comparable to those taken by a medical-grade EEG device under clinical settings.

The user device 122 can timestamp the data within the data collection software 126. Specifically, the collection software may do two things for timestamping: collect data from EEG device 132 with the exact time on it, and when the collection software administers a stimulus it generates a record of the event with a timestamp so that it can later be marked on the data, which has it's own time. The EEG data can be relayed in real time, but may have delay. The software used to collect this data can mitigate this problem using: i) a network time protocol that syncs the clocks of the EEG device 132 and user device 122, ii) clock drift compensation which estimates whether the two clocks are drifting apart and can re-align them, iii) buffering and latency measurement and correction, and iv) timestamps from source, when available, as they don't contain the delay.

The EEG passport generator 114 can process the EEG data (with synchronizations) to generate a set of EEG features. This set of features may generally be referred to as an EEG passport. The EEG passport generator 114 may use an automated pipeline to process the EEG data. The EEG passport can be generated using algorithms defined in a variety of EEG literature. The set of EEG features may include features from the EEG data itself, features from the association between the task content and the EEG data, features based on other features, etc. The EEG passport can be used to track brain health, and detect anomalous changes over time. The EEG passport may be stored (e.g., on the memory 106 of the server 102 or on the user device 122). The EEG passport may be stored with information such as the date of collection, a user ID, or other demographic information.

Different users have different brain patterns. Furthermore, the same disease (e.g., glioma) may present differently in different users. This creates a problem where no one feature of EEG data may be sufficient to provide a robust and comprehensive regime to detect the response or recurrence of, for example, a tumour in a population of patients. As is described further below, two patients (TGP002 and TGP003) had little overlap in the top 10 most variable features during lesion regression.

Accordingly, a system to diagnose and monitor a population of people may necessitate a robust array of EEG features to adequately account for the differences in the underlying biology and anatomy of the unique disease or conditions afflicting (or that may afflict) patients. The EEG passport generator 114 can overcome these challenges by generating a wide set of EEG features such as those presented in Table 1 above. The preponderance of features may help establish the detection of anomalies where the effect on any one feature is slight or the effect only presents in few features.

The anomaly detector 116 can detect anomalies in the EEG data. For example, the anomaly detector 116 may use the EEG passport to detect anomalies. The anomalies can include, for example, deviations between the data (e.g., the EEG passport) and expected data. In some embodiments, these deviations may include deviations from “healthy” EEG data. The “healthy” EEG data may be based on another user or other users. The “healthy” EEG data may be generated based on a subset of individuals with, for example, matching demographic data to that of the user. The “healthy” EEG data may be generated based on template “healthy” EEG data which has been modified to account for the user's specific demographic circumstances (e.g., comparing “healthy” EEG data of a typical adult to an older adult by increasing expected reaction time based on expected changes with age). In some embodiments, these deviations may include deviations from past EEG data from that same user (e.g., as compared to a past EEG passport). In some embodiments, the past data may include deviations from the last session or a statistical aggregation of several or all previous sessions. In some embodiments, the past data may include the first session (or a statistical aggregation of several initial sessions) to detect deviations that may be too gradual to identify on comparison with the last (or several recent) sessions. In some embodiments, the anomaly detector 116 can use one or more of the foregoing anomaly detection regimes (e.g., compare the EEG passport to “healthy” data and to past user data) or other further regimes. The anomaly detector 116 may use models trained using machine learning to detect anomalies within the EEG passport.

Based on the anomalies identified, the system 100 may provide a report. The report may be saved to the user's file, sent to the user, or sent to a third party (e.g., a physician). The system 100 may further be configured to determine and/or action next steps for the user. For example, based on the results of one neurocognitive task, the system 100 may instruct the patient to follow up with their physician. In other examples, the system 100 may be configured to carry out additional neurocognitive tasks based on the results of one or more initial neurocognitive tasks (e.g., to confirm the presence of an anomaly or to collect further information regarding the anomaly).

The memory 106 may generally follow the same description as for memory 1104 of FIG. 11 described below. The memory 106 may include task storage 118 and EEG passport storage 120. The functions achieved by the memory 106 may instead be carried out by the memory 126 of the user device 122 (e.g., the user device may store some of the past EEG passports).

The task storage 118 may store the tasks. For example, task storage 118 may store the audio and/or visual content to be provided to the user as part of the task. Task storage 118 may also include task metadata such as timing of various regions or instances within the task (e.g., regions where the user's eyes will be closed or the timing of an oddball stimuli). Tasks may include guidance for the user. Tasks may also include neurocognitive tasks which will probe the user to elicit specific EEG responses. The task presenter 110 may pull the task protocol from the task storage 118 and present the task to the user or cause the presentation of the task to the user.

The EEG passport storage 120 may store the EEG passport generated by the EEG passport generator 114. The anomaly detector 116 may retrieve one or more past EEG passports from the EEG passport storage 120 to detect anomalies in a current EEG passport over the past EEG passport(s). The EEG passport storage 120 may also store template EEG passports (e.g., “healthy” EEG passports) for use by anomaly detector 116 to detect anomalies in the EEG passport of the user.

The network interface 108 may generally follow the same description as for memory 1108 of FIG. 11 described below. The network interface 108 may operate to connect the server 102 with the user device 122 and/or the EEG device 132 through the network 150.

The user device 122 may be configured to present content to the user as part of, for example, a neurocognitive task. The user device 122 may be, for example, a mobile device or a laptop computer configured to access a platform server 102. In some embodiments, the user device may be configured to carry out one or more functions described above in connections with the server 102 (for example, the server 102 may send the user device 122 a copy of the requisite models and protocols and the user device 122 may be configured to carry out execution of the protocols using the models or that such protocols and models are stores on user device 122 and/or the user device 122 may store the tasks). The user device 122 includes a processor 124, a memory 126, a network interface 128, and an I/O interface 130.

The processor 124 may generally follow the same description as for processor 1102 of FIG. 11 described below. The processor may include an API 134 that may be configured to guide the user through a task (e.g., a neurocognitive task) at the same time that EEG data is being collected by the EEG device 132.

The memory 126 may generally follow the same description as for memory 1104 of FIG. 11 described below.

The network interface 128 may generally follow the same description as for network interface 1108 of FIG. 11 described below. The network interface 128 may operate to connect the user device 122 with the server 102 and/or the EEG device 132 through the network 150.

The I/O interface 130 may generally follow the same description as for I/O interface 1106 of FIG. 11 described below. The I/O interface 130 may interconnect with one or more output devices to deliver the task to the user. The I/O interface 130 may interconnect with a display screen to deliver visual content and/or a speaker to deliver audio content. Other output devices capable of delivery other output modalities are further possible.

The EEG device 132 may be, for example, a consumer-grade EEG device. The EEG device 132 may be configured to communicate with the user device 122 and/or the server 102 through the network 150. The EEG device 132 may collect EEG data from a user during the presentation of a neurocognitive task. In some embodiments, the system 100 may modify some or all of the processes carried out by the system 100 based on the type of EEG device 132 used. For example, the system 100 may require different types of EEG devices 132 to produce different standards of signal quality to pass before the task can begin (e.g., an EEG device 132 that can only produce low signal quality when working perfectly may be subject to lower signal quality standards than an EEG devices 132 that can produce high signal quality when working perfectly).

According to an aspect, there is provided a system 100 for decentralized electroencephalographic data collection across a plurality of neurocognitive tasks.

According to an aspect, there is provided a system 100 for remote and longitudinal monitoring of electroencephalographic changes. The system 100 includes a user interface application 134 for remotely collecting electroencephalographic data during a plurality of automated sessions that guides neurocognitive tasks while the electroencephalographic data is collected by an electroencephalographic device 132, the plurality of automated sessions over a plurality of time periods and a server that processes the electroencephalographic data using an automated pipeline to extract a plurality of features contained in the electroencephalographic data over the plurality of time periods, stores the features in a patient profile 120, generates a personal baseline using the electroencephalographic data using EEG passport generator 114, and performs anomaly detection in the profile of the plurality of features contained in the electroencephalographic data using anomaly detector 116. Each feature is associated with a stimuli of the audio and/or visual stimuli and a metric from the electroencephalographic data.

In some embodiments, the plurality of features are the plurality of features listed in Table 1.

In some embodiments, a feature in the set can be the mean signal value. This feature can be calculated by taking the average value of, for example, 2 seconds of data. The metric in this case may be the mean. This feature can contain the metric, but may be associated to an electrode and a stimuli (eyes open, oddball etc.).

In some embodiments, the user interface application 134 presents visual stimuli as part of the neurocognitive tasks.

In some embodiments, the user device 122 time-stamps the electroencephalographic data to synchronize it with the visual stimuli.

In some embodiments, the user device 122 uses the mean lag time to synchronize the electroencephalographic data and the timing of the presentation of the audio and/or visual stimuli

In some embodiments, the electroencephalographic device 132 is a consumer-grade electroencephalographic device.

In some embodiments, the system 100 further includes a display interface to display visual elements corresponding to detected anomalous changes.

According to an aspect, there is provided a system 100 for anomaly detection in remotely collected electroencephalographic data. The system 100 includes a measuring device 132 configured to capture electroencephalographic data comprising magnitudes of electric potentials over time in a scalp by using four or more discrete sensors and a user device 122 including a processor 124, non-volatile data storage 126, a screen, and audio speakers. The user device 122 including a user interface application 134 for guiding a user during an onboarding process and presenting audio and/or visual stimuli or continuous tasks as part of a neurocognitive task. The user device 122 is configured for wireless communication with the measuring device 132 to receive the magnitudes of the electric potentials over time in the scalp by using the four or more discrete sensors. The user device 122 is configured to synchronize the electroencephalographic data with the presentation of the audio and/or visual stimuli and/or the continuous tasks or transmit the electroencephalographic data to a remote server 102 for synchronization. Synchronization of the electroencephalographic data includes adding time-stamps and labels to the electroencephalographic data. The onboarding process includes checking pairing between the measuring device 132 and the user device 122, checking battery level of the measuring device 132, diagnosing quality of acquired signal in real time, and prompting the user to re-fit the four or more discrete sensors as needed.

In some embodiments, the screen includes at least one of a digital screen integrated into the user device or an external screen that the user device is in communication with.

In some embodiments, the user device 122 is one or more of a desktop computer, a laptop computer, a tablet or a smartphone.

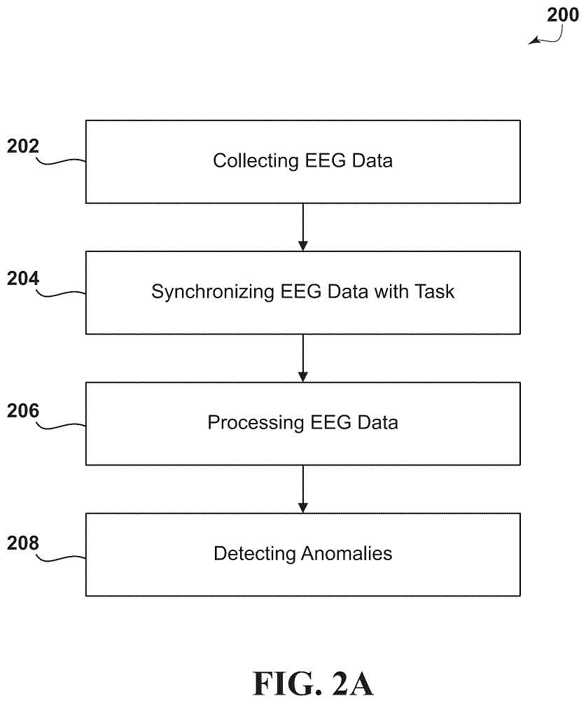

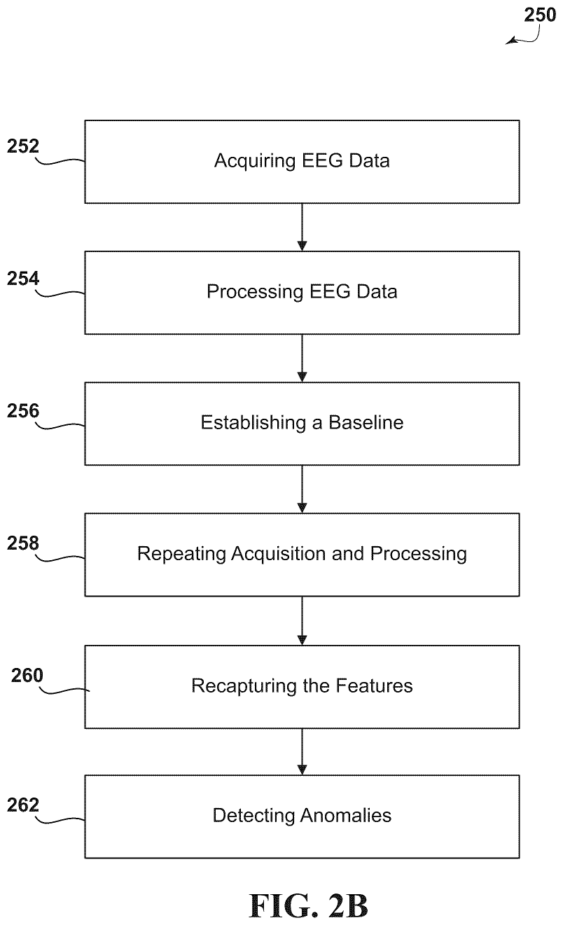

FIG. 2A shows a process diagram for a method 200 of collecting and processing EEG data, according to some embodiments.

The method 200 may enable remote and longitudinal monitoring of electroencephalographic changes. The method 200 may include collecting EEG data (block 202), synchronizing the EEG data with the task (block 204), processing the EEG data (block 206), and detecting anomalies (block 208).

Collecting EEG data (block 202) may include remotely collecting electroencephalographic data using, for example, an EEG device 132 during the presentation of a neurocognitive tasks involving a presentation of stimuli via, for example, a user device 122.

Synchronizing the EEG data with the task (block 204) may include synchronizing the electroencephalographic data to the presentation of the stimuli. For example, the EEG data may be time-stamped based on the stimuli that was being presented to the user.

Processing the EEG data (block 206) may include processing the electroencephalographic data using an automated pipeline to generate a profile of a plurality of features contained in the electroencephalographic data and a personal baseline. The profile may be, for example, an EEG passport.

Detecting anomalies (block 208) may include performing anomaly detection in the profile of the plurality of features contained in the electroencephalographic data. For example, an EEG passport may be compared to a “healthy” passport, past EEG passports, and/or a baseline EEG passport.

According to an aspect, there is provided a method 200 for remote and longitudinal monitoring of electroencephalographic changes. The method includes remotely collecting electroencephalographic data from an automated session of neurocognitive tasks involving a presentation of audio and/or visual stimuli, the automated session over a first time period (block 202), time synchronizing the electroencephalographic data to the presentation of the stimuli (block 204), processing the electroencephalographic data using an automated pipeline to extract a plurality of features contained in the electroencephalographic data for a patient profile (block 206), and performing anomaly detection in the profile of the plurality of features contained in the electroencephalographic data (block 208). The feature is associated with a stimuli of the audio and/or visual stimuli and a metric from the electroencephalographic data. The patient profile comprises of a personal baseline.

In some embodiments, the plurality of features are the plurality of features listed in Table 1.

In some embodiments, a feature in the set can be the mean signal value. This feature can be calculated by taking the average value of, for example, 2 seconds of data. The metric in this case may be the mean. This feature can contain the metric, but may be associated to an electrode and a stimuli (eyes open, oddball etc.).

In some embodiments, the method 200 further includes time-stamping the electroencephalographic data to synchronize the timing of the presentation of the audio and/or visual stimuli.

In some embodiments, the method 200 further includes using the mean lag time to synchronize the electroencephalographic data and the timing of the presentation of the audio and/or visual stimuli. In some embodiments, EEG data can be synchronized by measuring a metric of data transfer lag (such as the mean lag) for transmission and processing times and adjusting the lag to re-align the stimulus with the EEG data.

In some embodiments, processing the electroencephalographic data (block 206) includes extracting features comparable to other imaging modalities.

In some embodiments, the method 200 includes repeating the collecting and processing of electroencephalographic data over a plurality of time periods to establish the personal baseline for tracking and detecting changes in the features over the plurality of time periods, and to recapture the features over the plurality of time periods to compare the metrics over the plurality of time periods.

In some embodiments, the method 200 includes remotely collecting additional electroencephalographic data from another automated session of the neurocognitive tasks over a second time period and processing the additional electroencephalographic data using the automated pipeline to extract features from the additional electroencephalographic data, for comparison to the features from the first time period. The feature is associated with the same stimuli of the audio and/or visual stimuli and another metric from the electroencephalographic data.

In some embodiments, the method 200 includes storing, in memory, the profile of the plurality of features along with contextual information.

In some embodiments, the contextual information includes date of collection, a user identifier, and demographic data.

In some embodiments, the method 200 includes detecting habituation-dependent changes and environment-dependent changes in the electroencephalographic data.

In some embodiments, the method 200 includes providing an interactive signal quality check process prior to remotely collecting the electroencephalographic data. The interactive signal quality check provides real time feedback on electroencephalographic signal quality for the session. The interactive signal quality check is tuned to a specific device that generates the electroencephalographic data.

In some embodiments, the method 200 includes providing real time visual feedback on the electroencephalographic signal quality for the session.

In some embodiments, the method 200 includes detecting focal asymmetries in the electroencephalographic data. In some embodiments, the method 200 includes detecting substantially consistent focal asymmetries across many recordings in a row. In such embodiments, the consistent focal asymmetries may be used as a basis for detecting abnormalities.

In some embodiments, the method 200 includes remotely monitoring a diagnosed pathology over a plurality of time periods using electroencephalographic data.

In some embodiments, the method 200 includes remotely monitoring patient health over a plurality of time periods using electroencephalographic data.

In some embodiments, the method 200 includes tracking the same measurement using one or more features over a plurality of time periods.

In some embodiments, the method 200 includes measuring an improvement using the electroencephalographic data.

In some embodiments, the method 200 includes measuring a treatment response using the electroencephalographic data.

In some embodiments, the method 200 includes detecting a pathology using the electroencephalographic data.

In some embodiments, wherein processing the electroencephalographic data (block 206) includes identifying event-related potentials in the electroencephalographic data.

According to an aspect, there is provided a non-transitory computer readable medium having recorded thereon statements and instructions for execution by a processing system comprising at least one hardware processor to perform any one of the methods 200 described above.

FIG. 2B shows a process diagram for another method 250 of collecting and processing EEG data, according to some embodiments.

The method 250 may enable remote and longitudinal monitoring of electroencephalographic changes. The method 250 may include acquiring EEG data (block 252), processing the EEG data (block 254) establishing a baseline (block 256), repeating acquisition (block 252) and processing (block 254) (block 258), recapturing the features (block 260), and detecting anomalies (block 262).

Acquiring EEG data (block 252) may include remotely collecting electroencephalographic data using, for example, an EEG device 132 during the presentation of a neurocognitive tasks involving a presentation of stimuli via, for example, a user device 122.

Processing the EEG data (block 254) may include processing the electroencephalographic data using an automated pipeline to generate a profile of a plurality of features contained in the electroencephalographic data and a personal baseline. Each of the features may be a measurement of electroencephalographic signals that are associated with a position of one or more sensors from which the electroencephalographic data was acquired, and/or a visual and/or auditory stimulus and/or with a continuous task that is executed during acquisition of the electroencephalographic data. The profile may be, for example, an EEG passport. The features may include the features described in Table 1. The processing may be carried out on, for example, a user device 122 or on a remote server 102.

Establishing a baseline (block 256) may involve establishing a personal baseline for tracking and detecting changes in the features over subsequent sessions. The personal baseline may include a vector of weights of length equal to the number of features, and each of the weights may be a relative relevance assigned to the feature in the personal baseline. The baseline may be established on, for example, a user device 122 or on a remote server 102.

Repeating acquisition and processing (block 258) may include repeating the EEG acquisition using the EEG device 132 and processing the EEG data over a plurality of subsequent session.

Recapturing the features (block 260) may include recapturing the plurality of features over the plurality of sessions to compare the plurality of features over the plurality of sessions. The feature recapture may be carried out on, for example, a user device 122 or on a remote server 102.

Detecting anomalies (block 262) may include detecting anomalies in the plurality of features using any combination of one or more of the plurality of features (block 262). The detected anomaly may indicate presence or change of a medical condition. The feature recapture may be carried out on, for example, a user device 122 or on a remote server 102.

According to an aspect, there is provided a method 250 for anomaly detection in remotely collected electroencephalographic data. The method 250 includes acquiring electroencephalographic data (block 252), processing the electroencephalographic data using an automated pipeline to extract a plurality of features (block 254), establishing a personal baseline for tracking and detecting changes in the features over subsequent sessions (block 256), the personal baseline including a vector of weights of length equal to the number of features, and each of the weights are a relative relevance assigned to the feature in the personal baseline, repeating the acquisition step and processing step over a plurality of sessions (block 258), recapturing the plurality of features over the plurality of sessions to compare the plurality of features over the plurality of sessions (block 260), and detecting anomalies in the plurality of features using any combination of one or more of the plurality of features (block 262), the detected anomaly indicates presence or change of a medical condition. Each of the features are a measurement of electroencephalographic signals. Each of the features are associated with a position of one or more sensors from which the electroencephalographic data was acquired. The features are associated with a visual and/or auditory stimulus and/or with a continuous task that is executed during acquisition of the electroencephalographic data.

In some embodiments, the plurality of features are associated with an algorithm for processing the EEG signals.

In some embodiments, the plurality of features are the plurality of features listed in Table 1.

In some embodiments, the method 250 includes remotely collecting the electroencephalographic data from an automated session of neurocognitive tasks involving a presentation of the audio and/or visual stimuli and/or the continuous tasks, the automated session over a first time period, time synchronizing the electroencephalographic data to the presentation of the audio and/or visual stimuli and/or the continuous tasks, and processing the electroencephalographic data using the automated pipeline, either locally in an electronic device or remotely in a remote server.

In some embodiments, the medical condition is any type of tumour in the brain regardless of its cell-of-origin.

According to an aspect, there is provided a non-transitory computer readable medium having recorded thereon statements and instructions for execution by a processing system comprising at least one hardware processor to perform any one of the methods 250 described above.

### Embodiment Platform.

The following section relates to an embodiment platform, according to some embodiments. The following description is intended for illustrative purposes and does not limit the full scope of the concepts described herein.

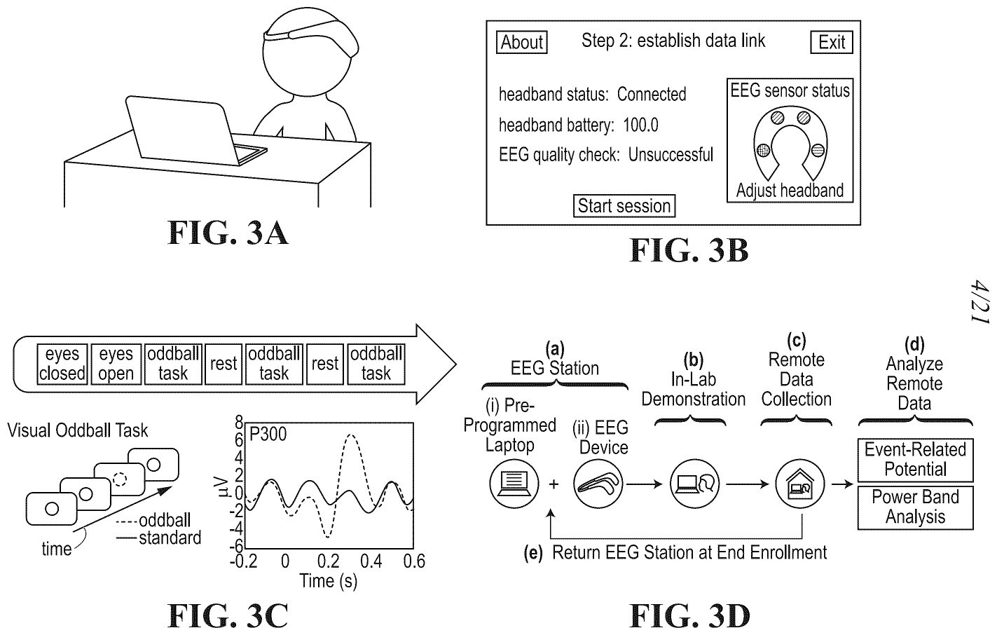

FIG. 3D illustrates a standard workflow for remote data collection using the embodiment platform, according to some embodiments. First, a laptop with software (a (i)) that can connect to and receive data from an EEG wearable (a (ii)) can be provided to a user. The user can, for example, attend an initial in-lab demonstration of the software to simultaneously perform EEG recordings with cognitive tasks (b) of interest. Longitudinal data collection can be done at home (c) with sessions as frequent as for example, weekly, bidaily, or even daily. Once collection is complete, or during regular hospital visits, devices can be returned to the lab for data retrieval and analysis (d).

The user may receive an introductory session to educate them on the embodiment platform. This may be delivered remotely or in-person. The user can, for example, receive training by a study coordinator on how to operate the consumer-grade EEG device and run the self-led recording sessions using the embodiment platform. The embodiment platform may provide a training module (or getting started). The user may be instructed to sit in a chair across from the laptop in a quiet room free of distractions.

FIG. 3A is a schematic showing a subject wearing the EEG wearable device while seated across a portable pre-programmed laptop with the embodiment platform, according to some embodiments.

Remotely, the users may perform EEG sessions recordings over a span of time (preferably in a quiet room free of distractions). The users may preferentially record EEG home sessions on the same days and around consistent times.

Upon opening the embodiment platform, the system may search for powered-on EEG devices, e.g., through wireless connections such as a Bluetooth connection. Once successfully connected, the system will display an interactive signal quality check, also displaying the power level, and connection status. To ensure the quality of the collected data, the session may only be executed when the user achieves a good EEG contact quality on all four electrodes.

FIG. 3B illustrates a user interface (UI) for an interactive initial signal quality check process, according to some embodiments. Colored circles can represent the status of each electrode (Green (diagonal lines): Accepted, low signal variability. Yellow (cross-hatched lines): Close to Accepted. Red (horizontal lines): Not Accepted, high signal variability.). To improve quality of remotely captured data, all circles may be required to be green to start the session.

The session task is chosen (preloaded), for example, in the embodiment platform from an editable JSON file. Once the session starts, audio commands can guide the subjects through the neurocognitive testing. The recorded EEG data can be written as the session progresses and can be stored in, for example, a CSV file and compiled to an SBL file for later analysis. The data can securely be saved anonymously on the local hard drive without, for example, ever passing through any proprietary third-party software. A feature of the embodiment platform is the modular nature that allows the central functionality to operate while being agnostic to the manufacturer of the wearable.

The embodiment platform may allow for customization and administration of neurocognitive tasks with simultaneous time-stamped EEG recordings compatible with the many current consumer-grade brain wearables. EEG can be recorded from, for example, a 4-channel dry electrode device. The embodiment platform can be sufficiently modular to allow for different EEG hardware systems to be integrated. Some consumer-grade EEG devices used can also be optimally balanced for cost and ease-of-use for working with complex populations such as patients with high-grade gliomas who may face varying degrees of cognitive impairments from surgery and chemoradiation therapy. The platform may take the EEG device into account when processing the data. For example, different EEG devices may produce different standards of signal quality to pass before the neurocognitive tasks can begin (e.g., devices that can only produce low signal quality when working perfectly may be subject to lower signal quality standards than a device that can produce high signal quality when working perfectly). In some embodiments, different EEG devices may permit different functionality (e.g., different neurocognitive tasks or different analyses) based on, for example, the obtainable signal quality.

The EEG session can include, for example, resting state task with closed and opened eyes (2 mins) because it is ubiquitous in clinical and research settings. These provide different basal EEG states to compare changes in background brain activity across time, acting as a personalized baseline. The first component of the sessions can be the resting task; first for, for example, 60 seconds with eyes-closed and then 60 seconds with eyes-open. During these components, users may be instructed to fix their eyes upon a cross in the center of the screen. The software may notify subjects when to close and open their eyes through audio commands.

The EEG session can also include an oddball task in which subjects can be shown a series of standard stimuli (e.g., green circle) and the oddball stimuli (e.g., blue circle). The task can be administered in three blocks consisting of, for example, 40 stimuli each, where each stimuli can randomly be assigned a color with a 90%/10% chance of being a standard and oddball respectively. Because the blue circles are less common, they often “surprise” the user's unconscious novelty seeking system which may be expected to elicit the commonly known P300 waveform. Each stimuli duration can randomly be determined to be between 800 ms and 1200 ms and the time between stimuli may also be randomly assigned between 300 ms and 500 ms after each stimuli to prevent timing between samples being predictable. Between the blocks of 40 stimuli, subjects can be given an unstructured 60-second rest period to prevent fatigue.

The EEG sampling frequency may be, for example, 256 Hz. EEG data can be analyzed using, for example, MNE-Python (MEG+EEG Analysis & Visualization) and filtered using an automated analysis pipeline with, for example, a bandpass filter between 0.50 Hz and 33.75 Hz with Hamming window. The data can be fragmented into, for example, 1 and 2-second epochs at stimuli events (e.g., oddball, standard, eyes-closed, eyes-open) precisely time-stamped by the embodiment platform. Artifacts-containing epochs above 80 uV can be rejected.

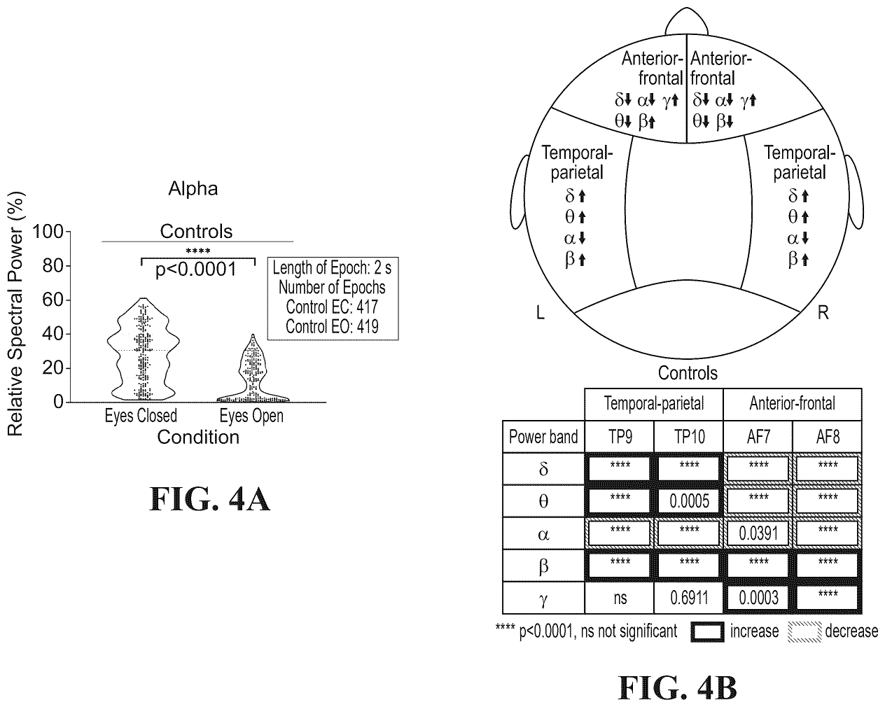

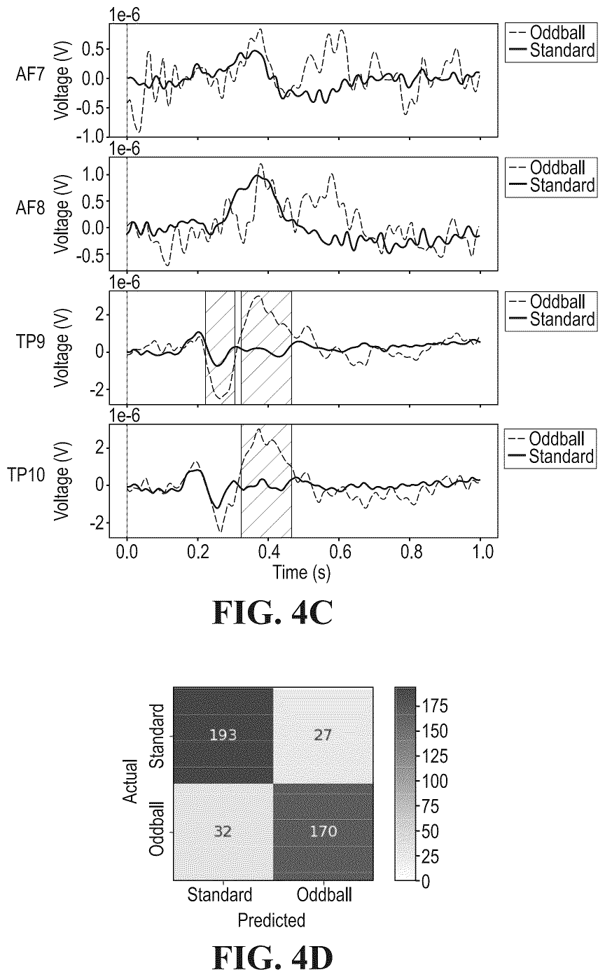

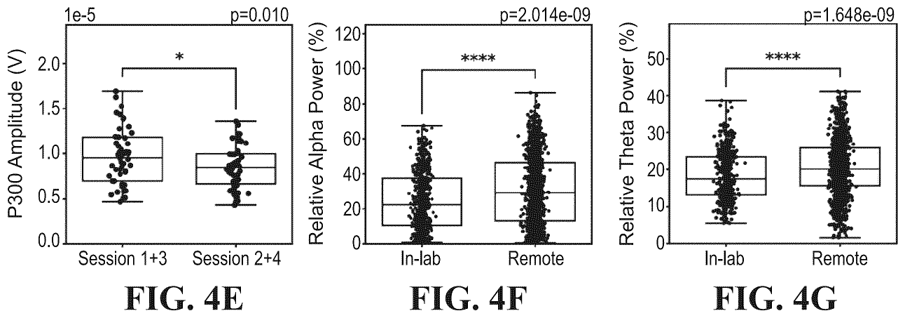

The evoked time courses for both the oddball and standard stimuli events can be plotted in each of the 4 channels to visually analyze the maximum peak amplitude in voltage between 200-400 ms (i.e., N200 and P300) and the amplitude and latency in the oddball peak in each channel can be measured (see, for example, FIG. 4C). A confusion matrix can be computed to evaluate the accuracy of a classification cross validation reported with each 1-second epoch as a sample between oddball and standard stimuli.

For each resting state condition, eyes-closed and eyes-open, the Fast Fourier Transform (FFT) and multitaper estimation power spectral analysis functions of each 2-second epoch can be performed. The power spectra as the area under the power spectrum density curve between 0.5 and 45 Hz as the total band power can be extracted and computed. The power band (e.g., delta, theta, alpha, beta, gamma) contribution may span across the frequency's range of interest (e.g., 8-12 Hz for alpha band) in each EEG channel. Relative band power can express the power in a frequency band as a percentage of the total power. Spectral markers such as Relative Power and Power Ratio Index may show potential in discriminating between healthy individuals and brain tumor patients (or other neurologically diseased patients).

FIG. 3C illustrates a sequence of neurocognitive tasks employed in a standard session (top), a diagram of visual oddball paradigm where oddball and standard stimuli are presented (bottom left), and a diagram sample of a typical P300 waveform (positive deflection occurring approximately around 300 milliseconds) evoked by the oddball stimuli (bottom right), according to some embodiments. The sequence of neurocognitive tasks employed in a standard session can include: resting state task in which subjects are to close or open their eyes for 1 minute each; and the visual oddball task in which subjects are shown a series of frequent ‘standard’ stimuli (e.g., green circle) and infrequent ‘oddball’ stimuli (e.g., blue circle).

To form the high-dimensional EEG-omic feature space, extracting power band features such as absolute power, power density, power ratios etc. may be carried out. Features corresponding to the amplitude and latency of each ERP may then be added. Measures of connectivity indexed with several different metrics like correlation, phase lag etc. can be extracted. This can lead to the generation of features which can be extracted.

Analysis can be conducted on a subject-wise basis given that the user may be heterogeneous, especially with respects to lesion location or other neurological factors, making it difficult to monitor consistent and uniform changes at the population level. T-tests can be used to identify candidate biomarkers that can distinguish patients from controls. Candidates may further be narrowed by selecting for a lower variation in the difference metrics. Ultimately, the feature shown may be selected manually from a handful of promising candidates.

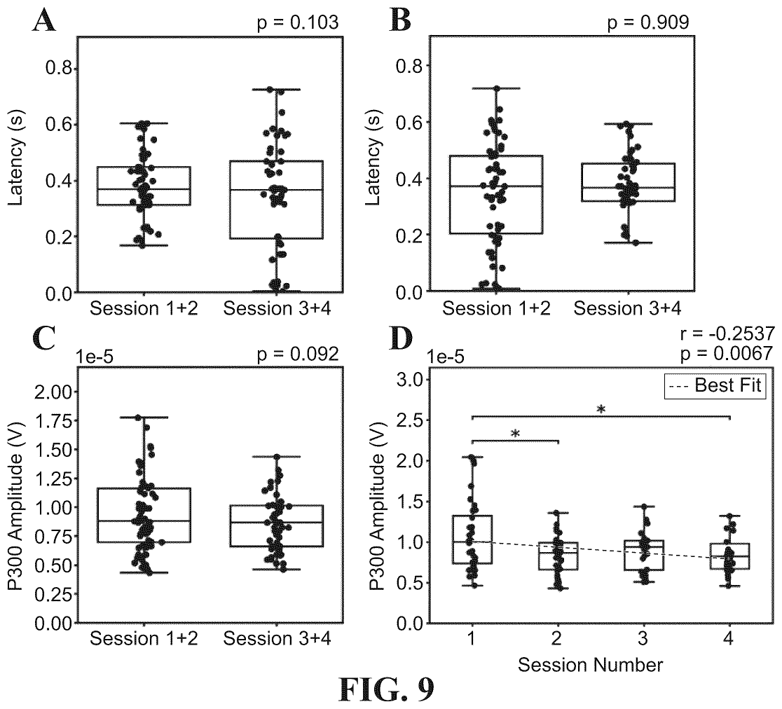

The statistical package SciPy statistics, MNE, and Prism 9.2.0 may be used for statistical analysis. Relative spectral power band values in eyes-closed and eyes-open conditions may be compared using one-way ANOVA for nonparametric evaluations to compare each frequency band across resting conditions in each of the four EEG channels. Nonparametric cluster-level paired t-tests may be used to evaluate significant clusters of differences between oddball and standard stimuli evoked responses. Pearson correlation may be obtained across the remote sessions and a corresponding p-value can be computed. One-tailed t-tests can be performed for the means of P300 amplitudes and latencies between the following combinations of remote sessions: 1 and 3 (first sessions of week) vs 2 and 4 (second sessions of week) (e.g., see FIG. 4E and FIG. 9B), sessions 1 and 2 (first week sessions) vs 3 and 4 (second week sessions) (e.g., see FIGS. 9A, and 9C), and all pairwise combination of the 4 sessions (e.g., see FIG. 9D). The outliers can be removed before comparisons using 1.5 times the interquartile range as the threshold. An additional one-tailed t-test can be used to evaluate power band values of 2-second epochs between in-lab and remote sessions.

Results from a Study Conducted Using an Embodiment Platform.

The following section relates to a proof-of-concept study performed with the embodiment platform described above. The following description is intended for illustrative purposes and does not limit the full scope of the concepts described herein.

The platform may provide a lightweight EEG testing platform that can be run on modest (for example, ˜$300) refurbished laptops and paired it with an EEG headset (˜$250) (FIG. 3A). Cohorts of geographically dispersed healthy cohorts and post-operative patients with high-grade gliomas were used to assess the reliability of the embodiment platform at collecting data across both resting and time-stamped electrophysiological data at participants' homes. In total, 20 young and healthy adults from three Canadian cities were recruited. Participants were first invited to receive a brief (˜20 minute) demonstration on how to connect and collect EEG data using the EEG headset device paired with a platform-installed laptop (FIG. 3B). Some embodiments of the platform may also allow the capture of time-locked EEG data synchronized to the presentation of visual stimuli presented to users (e.g. oddballs), to allow the collection of ERPs (FIG. 3C). To test the ability to remotely record EEG data, participants were then asked to take the hardware home and carry out a series of 4 additional EEG recording sessions across a nine-day period independent of any study operator (FIG. 3D).

For each subject, the study began with an introductory research session. The subjects were given a consumer-grade EEG device and a study laptop. To highlight the lightweight and low-cost nature of the developed system, refurbished laptops pre-programmed with the platform. The subjects received training by a study coordinator on how to operate the consumer-grade EEG device and run the self-led recording sessions using the embodiment platform. All subjects performed the first “in-lab session” accompanied by a study coordinator. The subjects were instructed to sit in a chair across from the laptop in a quiet room free of distractions. For all subjects, the introductory session was recorded between 9:00 AM and 5:00 PM.

23 healthy university-aged controls were recruited from various geographically distant Canadian cities. No controls exhibited a history of brain cancer. All controls provided consent for their participation.

Patients with newly diagnosed glioblastoma multiforme (GBMs (IDH-wildtype) were recruited after they underwent surgical resection of their tumor. In most cases, recruitment was done near the end of the patient's radiation treatment.

Following the introductory session in the research lab, the subjects took home the consumer-grade EEG device and a paired study computer to conduct the remainder of the sessions in a quiet environment in their own homes. Remotely, each healthy subject performed 4 EEG sessions recordings across 2 weeks, 2 home sessions each week that were similar to the initial introductory session. The home sessions were standardized to be recorded between 9:00 AM and 11:00 AM to control for the reported observation of elevated P300 amplitudes in the morning compared to the afternoon. Subjects were instructed to record EEG home sessions, on the same days and around consistent times. After the initial in-lab session, subjects were given the freedom to begin remote collection on their preferred day of the week. Subjects completed the collection at most within a 2-month timeframe from the date they were first enrolled.

In this study, a legacy software “lab version” was used with subjects enrolled from September to November 2023 which included the oddball task (n=14 and n=1 for controls and patients, respectively). The “home version” software includes both the oddball and resting state tasks that were implemented from November 2023 and onwards (n=12 and n=3 for controls and patients, respectively).

FIG. 4A shows paired violin plots showing a significant decrease in alpha activity between periods of eyes-closed and eyes-open in healthy controls (p<0.0001), according to some embodiments. Each point represents a 2-second extracted segment of the EEG data recorded from the left temporal parietal electrode (TP9).

FIG. 4B summarizes the significant changes in power spectra frequency bands changes from eyes-closed to eyes-open resting state conditions by brain regions in control subjects, according to some embodiments. Overall, controls showed symmetrical changes in brain activity. No significant differences detected between symmetrical electrodes in controls (see Table 2).

TABLE 2
P-values by Differences in Symmetrical Electrodes.

| Power band | Temporal-parietal | Anterior-frontal |
|---|---|---|
| δ | 0.9777 | 0.7523 |
| θ | 0.9997 | 0.9997 |
| α | 0.9998 | >0.9999 |
| β | 0.9856 | 0.6605 |
| γ | >0.9999 | >0.9999 |

**** p < 0.0001, ns not significant

No significant differences in power bands were detected between symmetrical electrodes (e.g., between TP9 and TP10, AF7 and AF8 electrodes) in controls.

FIG. 4C shows averaged ERP plots for each electrode derived from the combined data/ERPs of over 20 healthy controls, according to some embodiments. Amplitude is shown on y-axis in volts. Time is shown on x-axis in seconds. Clustered t-tests detected significant (red, p>0.05) differences between the oddball (dashed) and standard (solid) curves. Significant clusters resembling a N200 and P300 for TP9, and a P300 for TP10 were detected.

FIG. 4D shows single-epoch minimum-distance to the mean classifier performance of the oddball and standard stimuli, according to some embodiments. Single-epoch classifier performance (accuracy=0.860) confirmed oddball and standard stimuli are reliably different.

To first assess the overall fidelity of the embodiment platform to reliably capture baseline EEG data, the embodiment platform can guide participants through one-minute sessions of resting state in both open and closed eyes conditions (n=8 of 20 from healthy controls). Power band analysis highlighted the expected higher relative alpha power during periods of closed eyes (FIG. 4A: p<0.0001, n=8). Moreover, participants expressed expected symmetrical differences between eyes open and closed conditions on corresponding electrode positions on the left and right hemispheres (p-values reported in FIG. 4B). Together, these results support that embodiment platforms according to the present disclosure can remotely guide participants through a number of specific tasks and record annotated EEG data collected from an accessible consumer-grade device.

The precise time-stamping nature of the platform may offer the opportunity to carry out advanced neuro-cognitive measurement. Participants can be asked to perform a guided ERP visual-oddball task in which subjects were shown a series of frequent “standard” stimuli (green circle) and infrequent “oddball” stimuli (blue circle). By precisely time-stamping the recorded EEG data in the platform, distinct “P300” waveforms can be observed in the TP9 and TP10 electrodes when the infrequent oddball stimuli were presented (TP9 N200: p=0.002, TP9 P300: p=0.001, TP10 N200: p=0.311, P300: p=0.001; n=X participants; FIG. 4C). Additionally, using a minimum-distance-to-means classifier, the individual oddball and standard stimuli epoch can successfully be distinguished (accuracy: 86%; AUC=0.9438; random Chi square test: p=7.6e⁻⁴⁹) (FIG. 4D). Together, these results support that embodiment platforms according to the present disclosure can carry out fully automated time-stamped ERP experiments using low-cost EEG headsets even when independently carried out by remote subjects.

FIG. 4E-4G relate to habituation and accentuation of EEG signals in remote EEG recording.

FIG. 4E shows boxplots comparing the peak TP9 and TP10 amplitudes of sessions 1 and 3 (days 1 and 8) combined, and 2 and 4 (days 2 and 9) combined, according to some embodiments. Significant decreases in amplitude were observed in sessions 2 and 4 combined (p=0.010). P300 amplitude is shown on Y-axis in volts. Session number is shown on X-axis.

FIG. 4F shows relative alpha power between in-lab and remote recordings for eyes-closed condition in left temporal-parietal channel from controls (p<0.0001), according to some embodiments. Relative spectral power is shown on Y-axis in percent. Session environment is shown on X-axis.

FIG. 4G shows relative theta power between in-lab and remote recordings for eyes-closed condition in left anterior-frontal channel from controls (p<0.0001), according to some embodiments. Relative spectral power is shown on Y-axis in percent. Session environment is shown on X-axis.

FIG. 9A-9D shows session-to-session comparisons that showed habituation with a diminished P300 amplitude, according to some embodiments.

FIG. 9A shows boxplots comparing the latencies of each TP9 and TP10 amplitude peak from sessions 1 and 2 (days 1 and 2) combined, and 3 and 4 (days 8 and 9) combined, according to some embodiments. No significant differences were observed (p=0.103). Latency is shown on Y-axis in seconds. Session number is shown on X-axis.

FIG. 9B shows boxplots comparing the latencies of each TP9 and TP10 amplitude peak from sessions 1 and 3 (days 1 and 8) combined, and 2 and 4 (days 2 and 9) combined, according to some embodiments. No significant differences were observed (p=0.909). Latency is shown on Y-axis in seconds. Session number is shown on X-axis.

FIG. 9C shows boxplots comparing the peak TP9 and TP10 amplitudes of sessions 1 and 2 (days 1 and 2) combined, and 3 and 4 (days 8 and 9) combined, according to some embodiments. No significant differences were observed (p=0.092). P300 Amplitude is shown on Y-axis in volts. Session number is shown on X-axis.

FIG. 9D shows boxplots comparing the P300 peak amplitudes measured at the TP9 and TP10 electrodes for each remote session from all healthy participants, according to some embodiments. Amplitude is shown on Y-axis in volts. Session number is shown on X-axis. Significant decreases in amplitude between session 1 and session 2 (days 1 and 2), and session 1 and session 4 (days 1 and 9) were observed. Line of best fit is shown in red (p=0.0067, r=−0.2537). No significant differences were detected when similar analyses were performed on session latency (graph not shown).

The embodiment platform can detect longitudinal changes in the brain waves of healthy controls. This can be assessed by comparing how the study environment (in-lab vs remote) and repeated testing may affect EEG recordings. First, a slight negative correlation between session number and the P300 amplitude can be observed (r=0.2537, p=0.0067; see FIG. 9D). Session-to-session comparisons showed habituation with a diminished P300 amplitude when testing was performed on consecutive days (p=0.010; FIG. 4E). These results are in support of short habituation time frames in P300 amplitudes recorded using research-grade equipment. Comparison of relative power also revealed higher alpha and theta power in remote sessions during the eyes-closed condition when compared to in-lab recordings (p<0.0001 and p<0.0001; FIG. 4F-4G). This may be related to the increased relaxation participants felt in their more familiar remote environment. Overall, these experiments support that consumer-grade wearables, when coupled to appropriate software, have sufficient sensitivity to detect longitudinal changes in brainwaves and even more acute electrophysiological parameters that could serve as non-invasive and remotely collectable biomarkers for monitoring brain and disease state evolution.

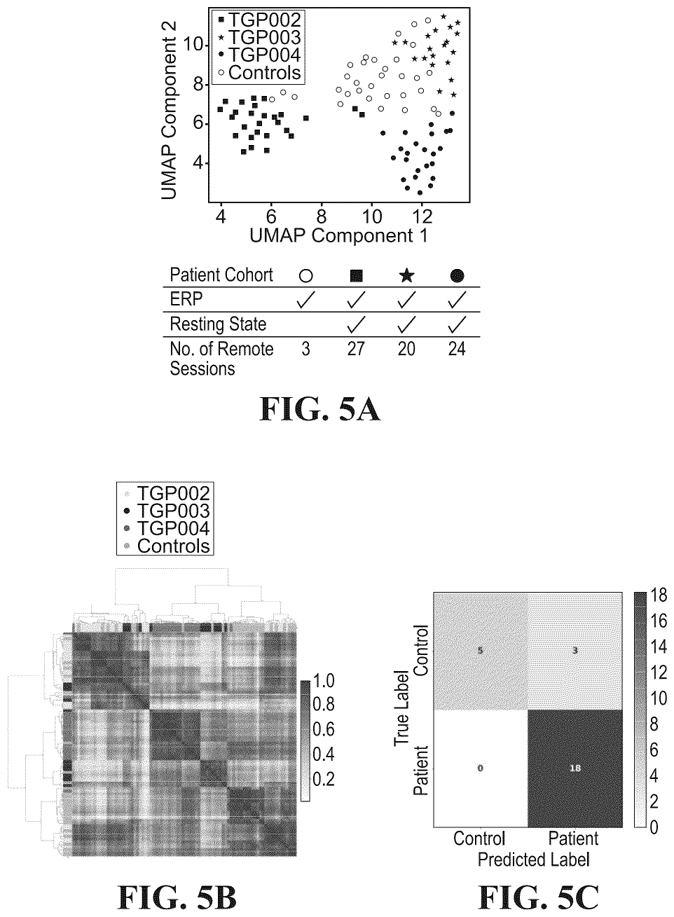

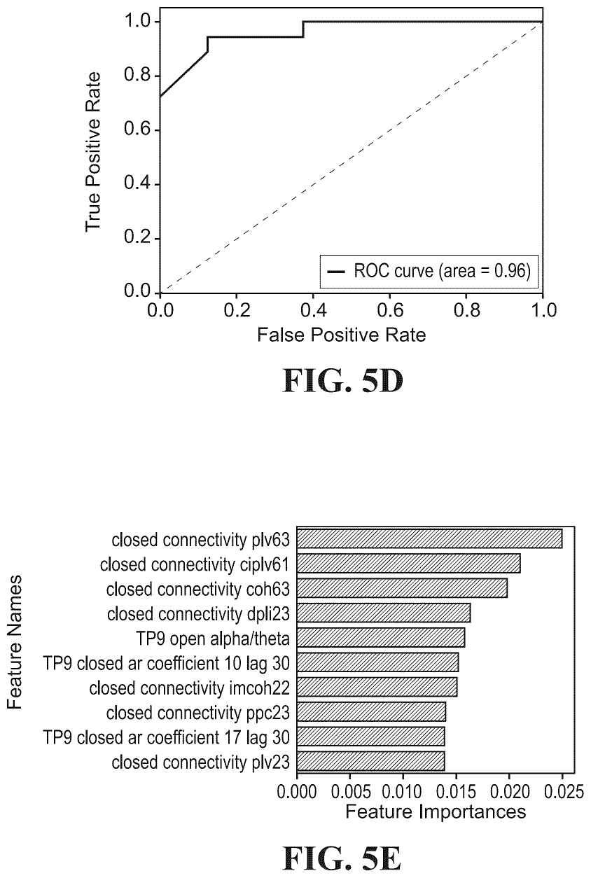

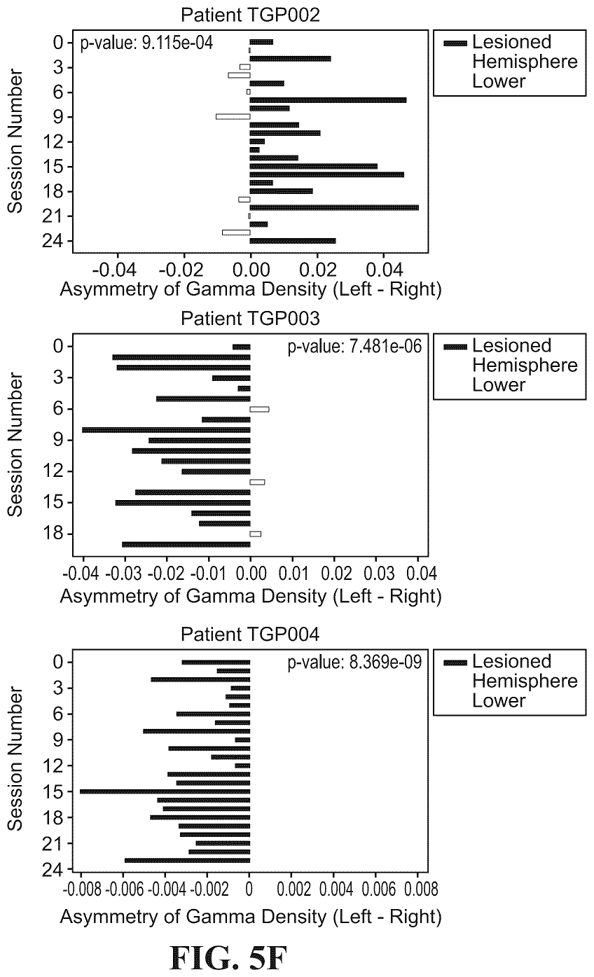

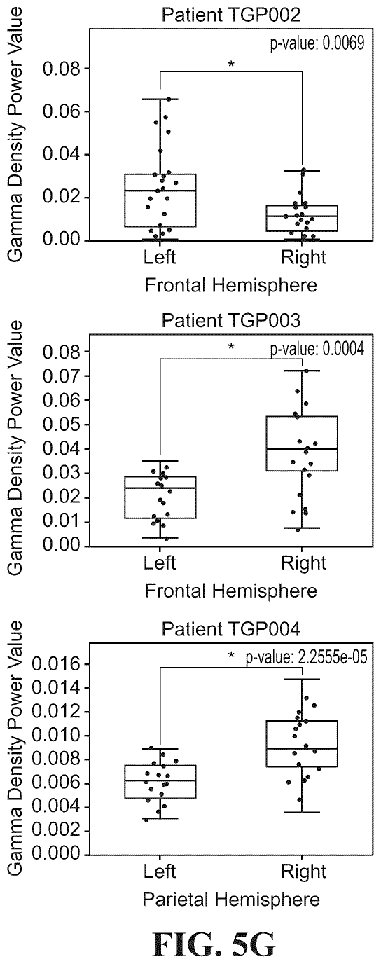

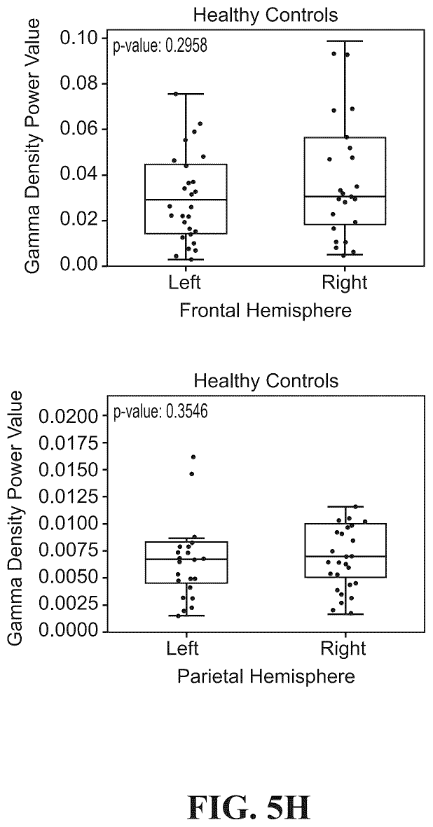

FIG. 5A shows a visualization of EEG-omic feature space for both patients and controls using UMAP dimensionality reduction, according to some embodiments.

FIG. 5B shows a heatmap showing hierarchical clustering of EEG-omic profiles, according to some embodiments.

FIG. 5C shows a confusion matrix showing proof-of-concept machine learning classification of patients with HGG and controls using a random forest with out-of-bag testing, according to some embodiments. Classification was tested against a chi-square even distribution (p<e−10).

FIG. 5D shows a receiver-operator characteristic curve for the classification described by FIG. 5C, according to some embodiments. The receiver-operator characteristic curve shows that the classifier is robust over a variety of classification thresholds (AUC=0.96).

FIG. 5E shows a histogram showing highest feature importance values identified by the random forest classifier, according to some embodiments.

FIG. 5F shows bar graphs showing consistent hemispheric-asymmetry of the gamma density across longitudinal sessions, according to some embodiments. Black bars indicate when the lesioned side was concordant with lower gamma power values.

FIG. 5G shows boxplots quantifying the significant asymmetrical decrease in gamma density on the lesional side, according to some embodiments.

FIG. 5H shows boxplots showing the symmetry (non-significance) in controls, according to some embodiments.

The ability to remotely capture symmetric EEG patterns from spatially separated electrodes on different parts of the skull, offers the potential to detect focal neurological changes concerning of intra-cranial pathology. To explore this prospect, a cohort of patients with high-grade gliomas (HGG) who were enrolled following their radiation therapy were recruited and remote monitoring performed on (n=4 patients; 74 total weekly recordings; Table 3).

TABLE 3
Characteristics of recruited patients with high-grade gliomas in the study to date.

| | | | | | | Enrolment | Last | No. |
|---|---|---|---|---|---|---|---|---|
| | | | | | | Start | Recording | of |
| | Age | | Pathological | Lesion | IDH1 | Date | Date | Remote |
| Case | (year) | Sex | diagnosis | location | (M/W) | (MM/YY) | (MM/YY) | Sessions |
| 1 | 35-40 | F | Diffuse | right | W | October 2022 | November 2022 | 3 |
| | | | Hemispheric | fronto- | | | | |
| | | | Glioma, H3 | parietal | | | | |
| | | | G34R mutant, | | | | | |
| | | | WHO Grade 4 | | | | | |
| 2 | 50-55 | F | Glioblastoma, | right | W | January 2023 | Ongoing | 27 |
| | | | IDH-wildtype, | frontal | | | | |
| | | | WHO Grade 4 | | | | | |
| 3 | 40-45 | M | Oligodendroglioma, | left | M | February 2023 | March 1924 | 30 |
| | | | IDH-mutated, | frontal | | | | |
| | | | 1p19q co-deleted, | | | | | |
| | | | WHO Grade 3 | | | | | |
| 4 | 50-55 | M | Astrocytoma, | left | M | February 2023 | April 1924 | 50 |
| | | | IDH-mutated, | parietal | | | | |
| | | | WHO Grade 3 | | | | | |
| 5 | 50-55 | F | Oligodendroglioma | right | NA | March 1924 | April 1924 | 17 |
| | | | OR | temporal | | | | |
| | | | cavernous | | | | | |
| | | | malformation | | | | | |
| | | | (TBD) | | | | | |
| 6 | 40-45 | F | Meningioma | left | NA | April 1924 | April 1924 | 4 |
| | | | | frontal | | | | |
| 7 | 20-25 | M | Cavernoma | Left | NA | May 1924 | Ongoing | NA |
| | | | (hemorrhagic | temporal | | | | |
| | | | lesion) | | | | | |

Sex: F female, M male, WHO World Health Organization, IDH isocitrate dehydrogenase, M mutant, W wildtype.
NA indicates information is not yet available.

Given the different stages, locations and clinical complexity of neurological disorders, a hypothesis agnostic pipeline can be developed that extracts approximately two thousand quantitative electrophysiological features (e.g., P300 parameters, power bands, connectivity metrics) from each session to generate an “EEG-omic” profile that can act as a passport for subject-specific longitudinal tracking. An unsupervised visualization of these EEG fingerprints on a Uniform Manifold Approximation and Projection (UMAP) shows that the EEG-omic profiles can reliably distinguish each patient from one another and the original control cohort (FIG. 5A) (K-mean silhouette score=0.4909). Similarly, unsupervised hierarchical clustering revealed individual EEG session formed clusters enriched in subject-specific recordings (FIG. 5B, cluster purity=83.5%) further supporting that remotely-collected EEG-omic profiles can serve as stable subject-specific profiles for disease and longitudinal analysis. Additionally, at the population level, random forest classification can distinguish patient recordings from those of the numerous healthy participants, demonstrating how data-driven approaches can leverage this EEG-omic passport to detect potentially subtle and complex biosignatures of disease (FIG. 5C-E accuracy=88.46%, AUROC=0.96, sensitivity=100%, specificity=62.5%). Specifically, asymmetric gamma density may be a stable and consistent abnormality detected in high-grade glioma patients post-surgery (FIG. 5F; binomial test p-values=9.115e−4, 7.481e−6, 8.369e−9). Importantly, subjects may show a significant and consistent reduction in gamma activity from session-to-session in the hemisphere ipsilaterally to the tumor and surgery (FIG. 5G; t-test: p-values=0.0069, 0.0004, 2.2555e−5). This feature can be symmetric in the EEG recordings performed in our healthy cohort (FIG. 5H; control p-values=0.2958, 0.3546). While this observation may partially be driven by the post-surgical context, the reduction in gamma may be used to remotely detect group-level and personalized focal biomarkers of pathology using consumer-grade EEG devices. Together, these results support that remotely recorded EEG profiles can retain spatially distinct information able to identify both group-level and patient-specific biosignatures of disease.

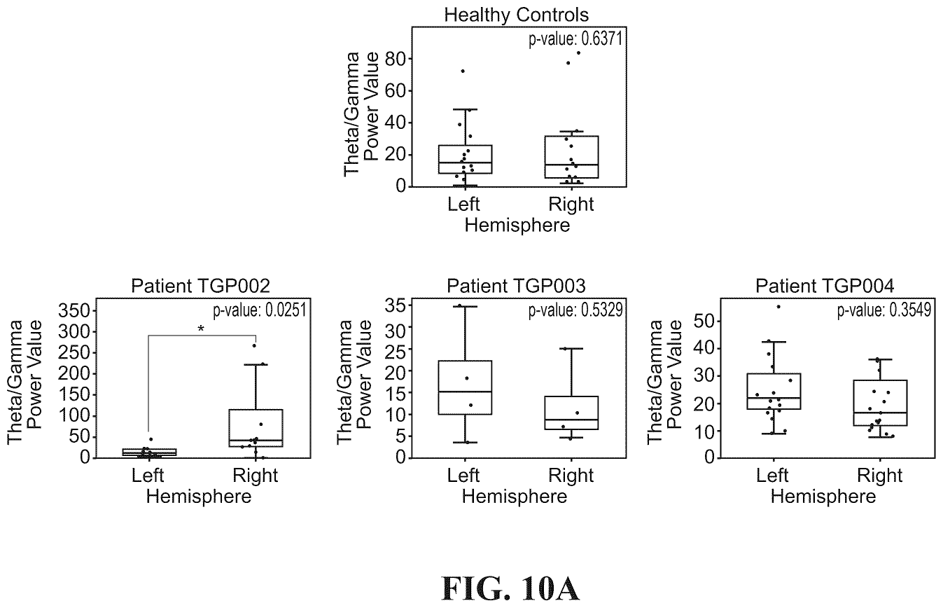

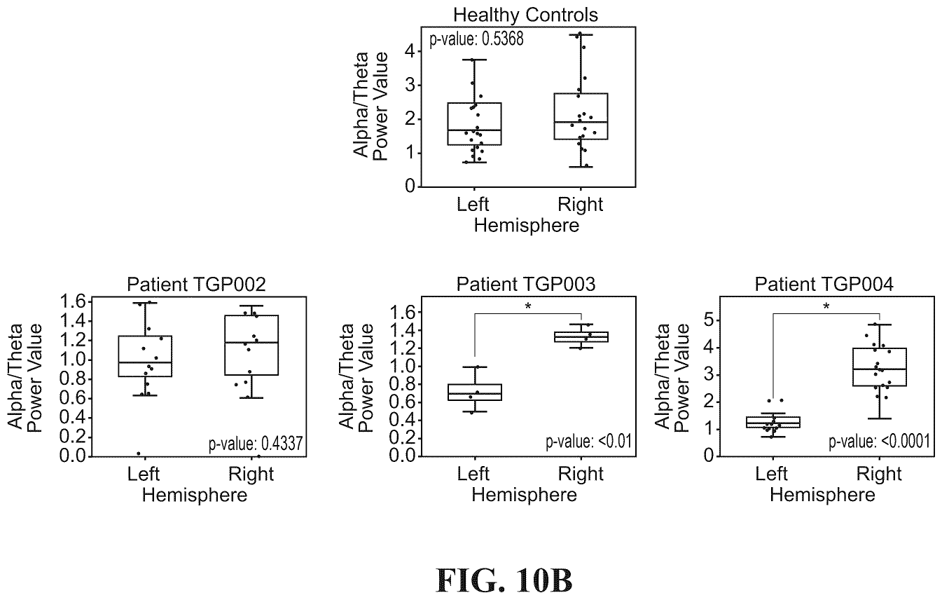

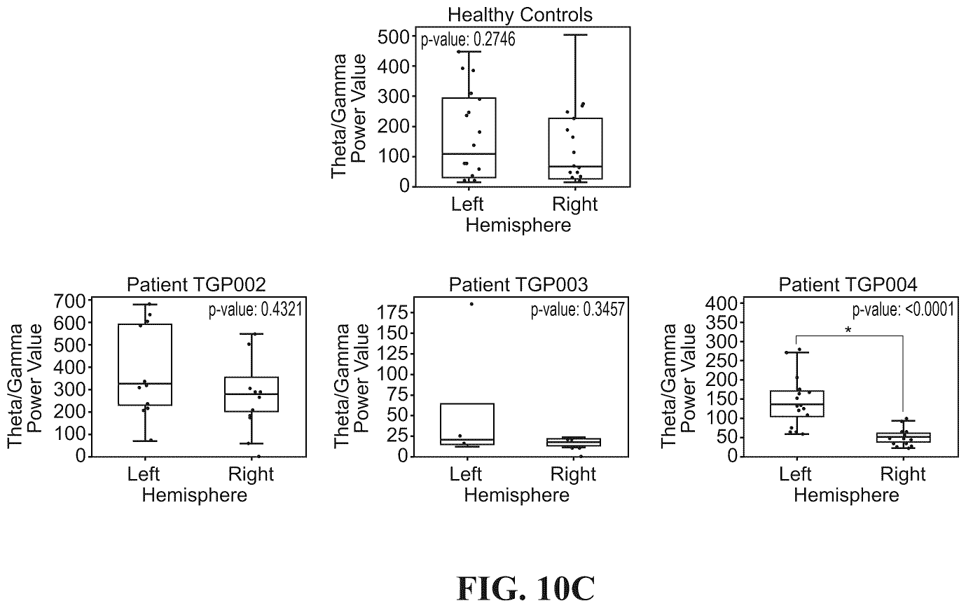

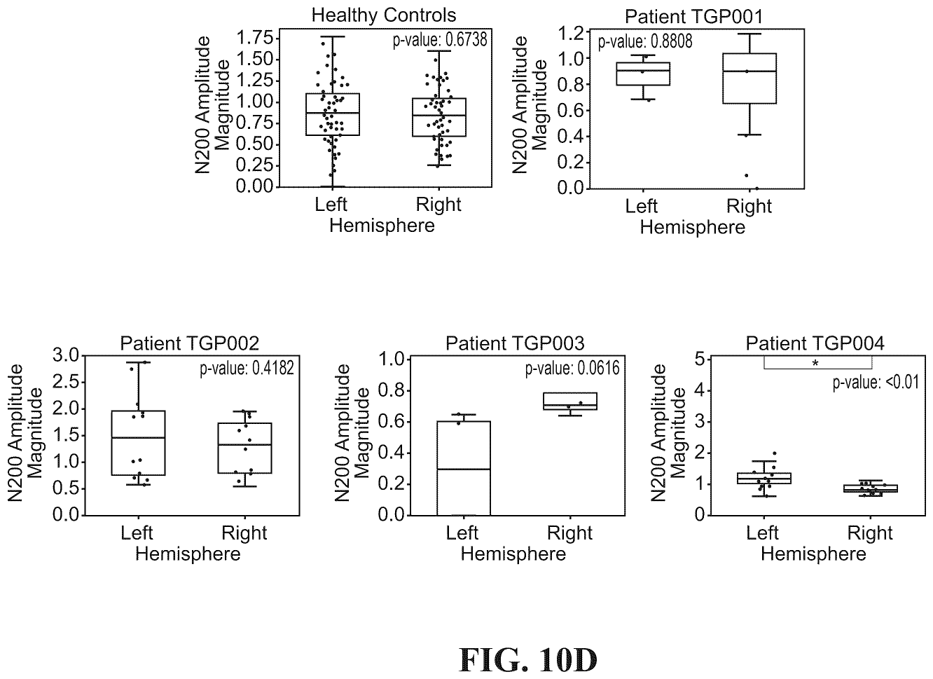

FIG. 10A-10D shows remote detection of differential and asymmetric EEG patterns shown in individual patients, according to some embodiments.

FIG. 10A shows boxplots showing significant differences of the parietal hemispheres' theta/gamma power ratio in patient TGP002 (p=0.0251) but not in healthy controls (p=0.6371), patient TGP003 (p=0.5329) or patient TGP004 (p=0.3549), according to some embodiments.

FIG. 10B shows boxplots showing significant differences in frontal hemispheres' alpha/theta power ratio in patient TGP003 (p<0.01) and patient TGP004 (p<0.0001) but not in the healthy controls (p=0.5368) or patient TGP002 (p=0.4337), according to some embodiments.

FIG. 10C shows boxplots showing significant differences of the frontal hemispheres' theta/gamma power ratio in patient TGP004 (p<0.0001) but not in healthy controls (p=0.2746), patient TGP002 (p=0.4321) or patient TGP003 (p=0.3457), according to some embodiments.

FIG. 10D shows boxplots showing significant differences in parietal hemispheres' N200 amplitude in patient TGP004 (p<0.01) but not in healthy controls (p=0.6738), patient TGP001 (p=0.8808), patient TGP002 (p=0.4182) or patient TGP003 (p=0.0616), according to some embodiments.

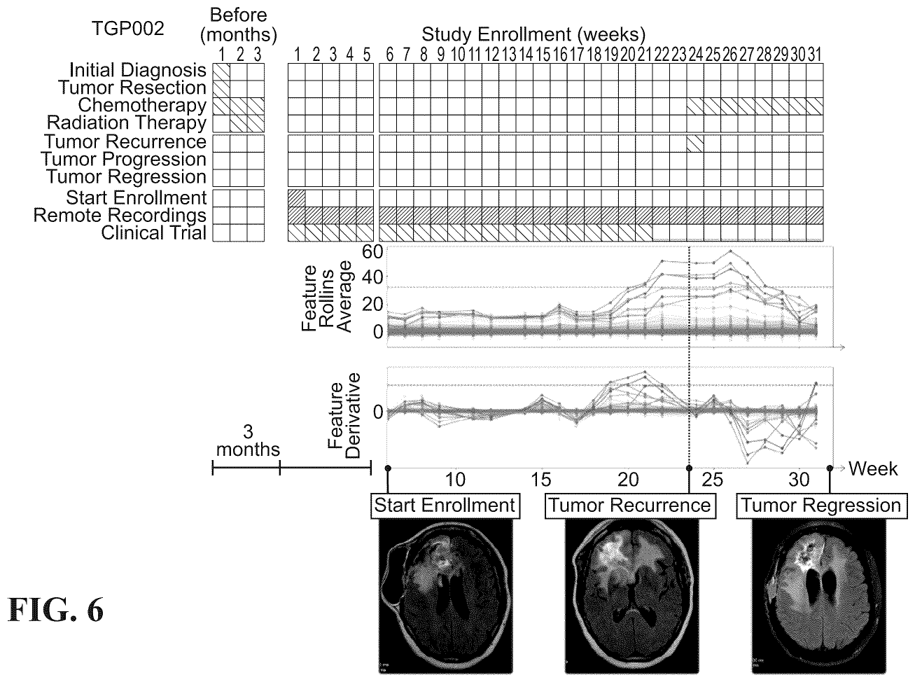

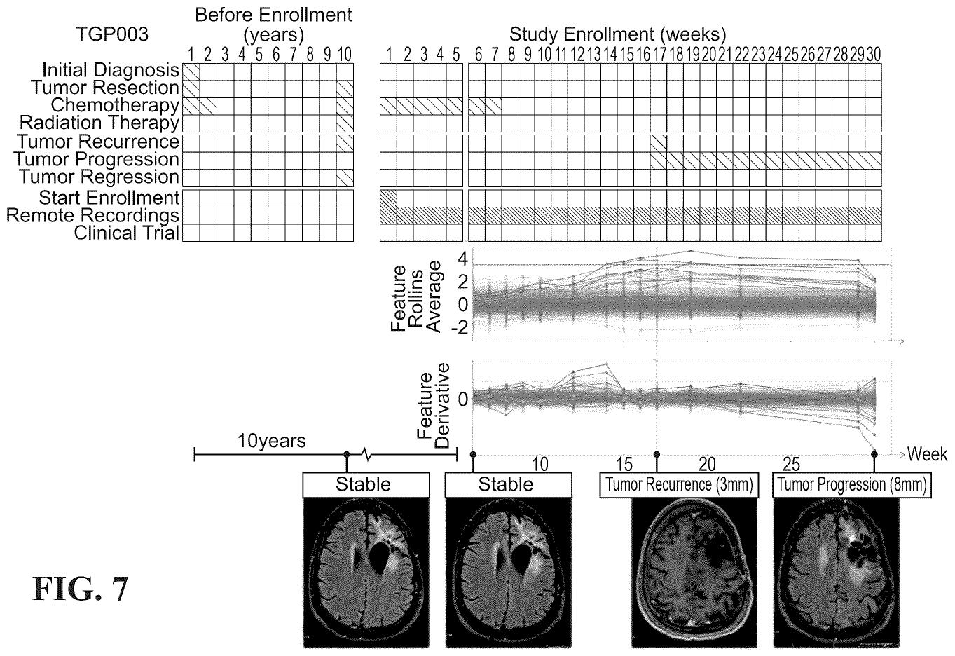

FIG. 6 shows timestamping unbiased and blinded radiological changes to the longitudinal monitoring for a patient (TGP002) with a recurrent tumor detected significant changes corresponding to biological tumor evolution, 24 weeks study enrolment elapsed, according to some embodiments. Final radiological impressions found in imaging reports define clinically significant changes, a significant increase (>1.5 full range (max-min) of early values from the mean) in the rolling average of features.

FIG. 7 shows identified individual spatiotemporal changes in features from longitudinal monitoring a patient (TGP003) corresponding to biological tumor evolution, ˜17 weeks study enrolment elapsed (enrolment was longer than 17 weeks but there was not 100% adherence to protocol so they might have ended up with 17 sessions), according to some embodiments. Numerous EEG features in the left frontal electrode (TP9) congruent with the appearance of an enhancing and growing nodule (p-value=0.006).

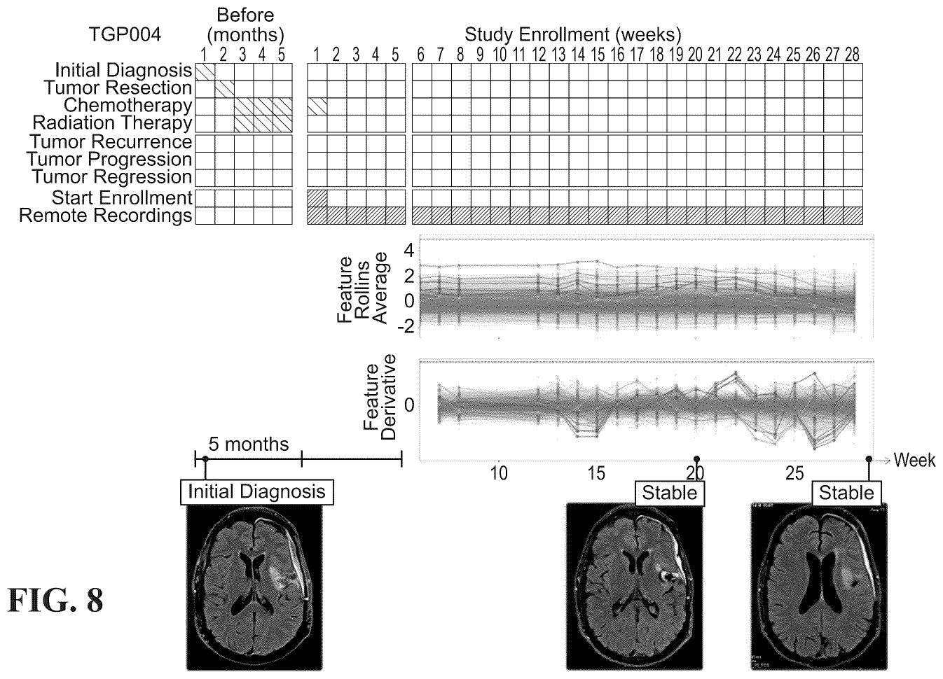

FIG. 8 shows alignment of EEG collected from consumer-grade wearables and medical-grade MRI scans by longitudinal monitoring of a patient (TGP004), according to some embodiment. The figure shows no significant changes corresponding to biological evolution. Substantial longitudinal data did not show any definitive radiological indication of recurrence confirmed using quarterly MRI surveillance scans.

The rapid and inevitable recurrence of many high-grade gliomas can offer an opportunity to remotely capture evolutions in patient-specific EEG profiles and compare them with other existing imaging modalities. The temporal trajectories of each individual feature within the EEG-omic signature with radiologic evidence of recurrence using patients' quarterly MRI surveillance scans was mapped and cross-referenced (FIG. 6-8). For this, the final radiological impressions found in the patients imaging report can be used to define clinically significant changes. Through this approach, two of our three enrolled patients with resting state data were identified as having focal contrast-enhancing regions indicative of potential tumor recurrences or progression. By timestamping these unbiased and blinded radiological changes to longitudinal data, several changes in individual EEG-omic features can be identified that had concordant spatiotemporal patterns. For example, in patient TGP002 a significant increase (>1.5 full range (max-min) of early values from the mean) in the rolling average of a number of features in the frontal electrodes was found (FIG. 6; p-value=0.020). This location coincided with a substantial increase in edema in the brain region beneath this specific electrode. Notably, these values showed a subsequent trend downwards that was consistent with radiological response and regression of the lesion to palliative chemotherapy. Similarly, in patient TGP003, focal large time congruent changes were found (>1.5 full range of early values) in numerous EEG features in the left frontal electrode with the appearance of an enhancing and growing nodule (FIG. 7 p-value=0.006). Notably, little overlap was observed in the top 10 most variable features in these two patients supporting a potential personalized nature of changes that may be driven by differences in the underlying biology and anatomy of these lesions. Interestingly, the final patient within the cohort (patient TGP004) that captured substantial longitudinal data did not show any definitive radiological indication of recurrence (as of the preparations of this application), and likewise showed no substantial changes in rolling average of session-to-session recording across the entire recording period (FIG. 8). Overall, this relatively dense longitudinal collection of data highlights the potential close alignment of EEG changes collected from consumer-grade wearables and medical grade MRI surveillance scans. Moreover, they highlight the improved sensitivity and personalized nature offered by recording combinatorial EEG features from multiple locations across the skull.

Brain wearables have the potential to capture large volumes of EEG data outside of traditional research and health care environments. An EEG platform that connects consumer-grade EEG devices to time-stamped data recordings and reliability of an embodiment platform benchmarked across several relevant scenarios in healthy participants is herein described. Importantly, the embodiment platform can be run on modest refurbished laptops and can be easily customized and expanded to administer additional automated neurocognitive tasks. As a proof-of-concept for the biomedical potential of remote EEG, an embodiment platform was deployed to collect weekly longitudinal recordings in a cohort of three patients with HGG. The aggressive nature of high-grade gliomas may offer a unique ability to track EEG recordings prospectively as the disease evolves. Results highlight that these consumer-grade EEG wearables can capture both group-level and individualized biosignatures that are consistent across repeated measures, distinguished from controls, and may longitudinally evolve in correspondence to radiological changes. The platform may also be used to track the evolution of brain tumors in, for example, larger brain tumor cohorts. Given the relatively high adherence to weekly recording within the sensitive cohort, this platform and experimental setup may be easily adapted and applied to other pathophysiological states and/or healthy states to facilitate non-invasive and real-time monitoring of brain development and disease evolution.

### Computational Implementation.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

FIG. 11 is a schematic diagram of computing device 1100, according to some embodiments. As depicted, computing device 1100 includes at least one processor 1102, memory 1104, at least one I/O interface 1106, and at least one network interface 1108. The computing device components may be connected in various ways including directly coupled, indirectly coupled via a network, and distributed over a wide geographic area and connected via a network (which may be referred to as “cloud computing”).

For simplicity only one computing device 1100 is shown but the system may include more computing devices 1100 operable by users to access remote network resources and exchange data. The computing devices 1100 may be the same or different types of devices.

For example, and without limitation, the computing device 1100 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Each processor 1102 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1104 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 1106 enables computing device 1100 to interconnect with one or more input devices, such as a keyboard, mouse, camera, touch screen and a microphone, or with one or more output devices such as a display screen and a speaker.

Each network interface 1108 enables computing device 1100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 1100 is operable to register and authenticate users (using a login, unique identifier, and password for example) prior to providing access to applications, a local network, network resources, other networks and network security devices. Computing devices 1100 may serve one user or multiple users.

### General Implementation Details.

Applicant notes that the described embodiments and examples are illustrative and non-limiting. Practical implementation of the features may incorporate a combination of some or all of the aspects, and features described herein should not be taken as indications of future or existing product plans. Applicant partakes in both foundational and applied research, and in some cases, the features described are developed on an exploratory basis.

The following discussion provides many example embodiments. Although each embodiment represents a single combination of inventive elements, other examples may include all possible combinations of the disclosed elements. Thus, if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only.

What is claimed is:

1. A method for remote and longitudinal monitoring of electroencephalographic changes, the method comprising:
remotely collecting electroencephalographic data from an automated session of neurocognitive tasks involving a presentation of audio and/or visual stimuli, the automated session over a first time period;
time synchronizing the electroencephalographic data to the presentation of the stimuli;
processing the electroencephalographic data using an automated pipeline to extract a plurality of features contained in the electroencephalographic data for a patient profile, wherein a feature is associated with a stimuli of the audio and/or visual stimuli and a metric from the electroencephalographic data, wherein the patient profile comprises of a personal baseline; and
performing anomaly detection in the profile of the plurality of features contained in the electroencephalographic data.

2. The method of claim 1, wherein the plurality of features are the plurality of features listed in Table 1.

3. The method of claim 1, time synchronizing comprises at least one of time-stamping the electroencephalographic data to synchronize the timing of the presentation of the audio and/or visual stimuli, and using the mean lag time to synchronize the electroencephalographic data and the timing of the presentation of the audio and/or visual stimuli.

4. The method of claim 1 further comprising:
remotely collecting additional electroencephalographic data from another automated session of the neurocognitive tasks over a second time period;
processing the additional electroencephalographic data using the automated pipeline to extract features from the additional electroencephalographic data, for comparison to the features from the first time period, wherein a feature is associated with the same stimuli of the audio and/or visual stimuli and another metric from the electroencephalographic data.

5. The method of claim 1 further comprising:
storing, in memory, the profile of the plurality of features along with contextual information, wherein the contextual information comprises date of collection, a user identifier, and demographic data.

6. The method of claim 1 further comprising:
detecting habituation-dependent changes and environment-dependent changes in the electroencephalographic data.

7. The method of claim 1 further comprising:
detecting focal asymmetries in the electroencephalographic data.

8. The method of claim 1 further comprising: remotely monitoring at least one of a diagnosed pathology and patient health over a plurality of time periods using electroencephalographic data.

9. The method of claim 1 further comprising: tracking the same measurement using one or more features over a plurality of time periods.

10. The method of claim 1 further comprising: measuring at least one of an improvement and a treatment response using the electroencephalographic data.

11. The method of claim 1 further comprising: detecting a pathology using the electroencephalographic data.

12. The method of claim 1, wherein processing the electroencephalographic data comprises identifying event-related potentials in the electroencephalographic data.

13. A system for remote and longitudinal monitoring of electroencephalographic changes, the system comprising:
a user interface application for remotely collecting electroencephalographic data during a plurality of automated sessions that guides neurocognitive tasks while the electroencephalographic data is collected by an electroencephalographic device, the plurality of automated sessions over a plurality of time periods; and
a server with at least one hardware processor and memory, wherein the server processes the electroencephalographic data using an automated pipeline to extract a plurality of features contained in the electroencephalographic data over the plurality of time periods, stores the features in a patient profile, wherein a feature is associated with a stimuli of the audio and/or visual stimuli and a metric from the electroencephalographic data, generates a personal baseline using the electroencephalographic data; and performs anomaly detection in the profile of the plurality of features contained in the electroencephalographic data.

14. The system of claim 13, wherein the plurality of features are the plurality of features listed in Table 1.

15. The system of claim 13, wherein the user device synchronizes the electroencephalographic data and the timing of the presentation of the audio and/or visual stimuli by at least one of time-stamping the electroencephalographic data and using the mean lag time.

16. The system of claim 13, wherein the electroencephalographic device is a consumer-grade electroencephalographic device.

17. A method for anomaly detection in remotely collected electroencephalographic data, the method comprising:
acquiring electroencephalographic data;
processing the electroencephalographic data using an automated pipeline to extract a plurality of features, wherein each of the features are a measurement of electroencephalographic signals, wherein each of the features are associated with a position of one or more sensors from which the electroencephalographic data was acquired, wherein the features are associated with a visual and/or auditory stimulus and/or with a continuous task that is executed during acquisition of the electroencephalographic data;
establishing a personal baseline for tracking and detecting changes in the features over subsequent sessions, wherein the personal baseline comprises a vector of weights of length equal to the number of features, and each of the weights are a relative relevance assigned to the feature in the personal baseline;
repeating the steps (a) and (b) over a plurality of sessions;
recapturing the plurality of features over the plurality of sessions to compare the plurality of features over the plurality of sessions;
detecting anomalies in the plurality of features using any combination of one or more of the plurality of features, and wherein the detected anomaly indicates presence or change of a medical condition.

18. The method of claim 17, wherein the plurality of features are associated with an algorithm for processing the EEG signals.

19. The method of claim 17, wherein the plurality of features are the plurality of features listed in Table 1.

20. The method of claim 17, further comprising:
remotely collecting the electroencephalographic data from an automated session of neurocognitive tasks involving a presentation of the audio and/or visual stimuli and/or the continuous tasks, the automated session over a first time period;
time synchronizing the electroencephalographic data to the presentation of the audio and/or visual stimuli and/or the continuous tasks;
processing the electroencephalographic data using the automated pipeline, either locally in an electronic device or remotely in a remote server.
